(19) United States
(12) Patent Application Publication
(10) Pub. No.: US 2013/0218054 A1
(43) Pub. Date: Aug. 22, 2013

(54) SEPARATION DEVICE FOR ULTRASOUND ELEMENT

(71) Applicants: Ariel Sverdlik, Tel-Aviv (IL); Or Shabtay, Kibbutz Farod (IL)

(72) Inventors: Ariel Sverdlik, Tel-Aviv (IL); Or Shabtay, Kibbutz Farod (IL)

(73) Assignee: CardioSonic Ltd., Tel-Aviv (IL)

(21) Appl. No.: 13/880,124

(22) PCT Filed: Oct. 18, 2011
(86) PCT No.: PCT/IB11/54638

§ 371 Date: Apr. 18, 2013

Related U.S. Application Data
(60) Provisional application No. 61/393,947, filed on Oct. 18, 2010.
(60) Provisional application No. 61/453,234, filed on Mar. 16, 2011.

(51) Int. Cl. A61N 7/00

(52) U.S. Cl. CPC A61N 7/00
USPC 601/2

(57) ABSTRACT

There is provided in accordance with an exemplary embodiment of the invention a method and for keeping an ultrasound emission element away from a wall of a blood vessel or cavity comprising: inserting a catheter comprising the ultrasound emission element into a blood vessel or body cavity such that the ultrasound emission element is prevented from contacting a wall of the blood vessel or the cavity. There is also provided in accordance with an exemplary embodiment of the invention a device for keeping an ultrasound emission element away from a wall of a blood vessel or lumen.

### RELATED APPLICATIONS

This is a PCT application which claims the benefit of priority of U.S. Provisional Patent Applications No. 61/393,947 filed Oct. 18, 2010, and No. 61/453,234 filed Mar. 16, 2011, the contents of which are incorporated herein by reference in their entirety.

The present application is related to co-filed, co-pending and co-assigned patent applications entitled:

“THERAPEUTICS RESERVOIR” (attorney docket no. 52341), which teaches an apparatus and a method for forming a drug reservoir as a possible application of the ultrasound energy application described herein;

“AN ULTRASOUND TRANSCEIVER AND CONTROL OF A THERMAL DAMAGE PROCESS” (attorney docket no. 52342), which teaches an apparatus and method for performing ultrasonic imaging, such as to provide feedback about the effect of treatment on tissues as described herein;

“ULTRASOUND EMISSION ELEMENT” (attorney docket no. 52344), which teaches an apparatus for generating relatively high intensity ultrasound, such as to apply energy to cause the desired effects in tissue as described herein;

“AN ULTRASOUND TRANSCEIVER AND USES THEREOF” (attorney docket no. 52345), which teaches a method for feedback and control of the ultrasonic emission element, such as to use the same ultrasonic element for treatment and imaging, potentially useful when treating and imaging as described herein; “AN ULTRASOUND TRANSCEIVER AND COOLING THEREOF” (attorney docket no. 52346), which teaches a method for cooling of the ultrasonic emission element, potentially useful when applying energy as described herein; and

“TISSUE TREATMENT” (attorney docket no. 52347), which teaches a method of selectively targeting and treating tissues using ultrasound, potentially useful when applying energy as described herein;

the disclosures of which are incorporated herein by reference.

### FIELD AND BACKGROUND OF THE INVENTION

The present invention, in some embodiments thereof, relates to an ultrasound treatment system and, more particularly, but not exclusively, to a device for maintaining at least a distance between an ultrasound emission element and a tissue.

Sverdlik et al, in PCT/IL2008/000234, filed Feb. 21, 2008 disclose: “Described is a method of stabilizing blood vessel wall abnormality. The method includes ultrasonically heating at least a portion of the blood vessel wall having the abnormality; monitoring a parameter related to a property of at least a portion of the to heated portion of the blood vessel wall; and stopping the heating when the monitored parameter changes by a predetermined factor or after the monitored parameter changes in a slow enough rate.”

Additional background art includes:

EP 1769759

U.S. Pat. No. 5,699,804

U.S. Pat. No. 7,410,486

U.S. Pat. No. 7,621,929

U.S. Pat. No. 7,717,948

U.S. Pat. No. 7,771,372

US patent application 2008228111

US patent application 2009216246

US patent application 2010091112

### SUMMARY OF THE INVENTION

An aspect of some embodiments of the invention relates to a separation device to provide at least a minimal distance between an ultrasound emission element on a catheter and a tissue, non-limiting examples of tissue include a wall of a blood vessel and/or cavity. Optionally, the distance is controllable and/or adjustable. Optionally, the distance is constant. Alternatively, the distance is variable. In an exemplary embodiment of the invention, the separation device provides for at least some volume of blood flow over the element, for example, between the element and the wall.

There is provided in accordance with an exemplary embodiment of the invention a method for keeping an ultrasound emission element, for example a transducer, away from a wall of a blood vessel or cavity comprising: inserting a catheter comprising a ultrasound emission element into a blood vessel or body cavity such that the ultrasound emission element is prevented from contacting a wall of the blood vessel or the cavity.

In an exemplary embodiment of the invention, the method comprises preventing by a part of the catheter.

In an exemplary embodiment of the invention, the method comprises maintaining at least a minimum distance between the ultrasound emission element and the wall. Optionally, the distance is constant. Optionally or alternatively, the distance is adjustable. Optionally or additionally, the distance is variable.

There is provided in accordance with an exemplary embodiment of the invention an apparatus for keeping an ultrasound emission element away from a wall of a blood vessel or cavity comprising: an extension on a catheter comprising an ultrasound emission element, the extension configured to prevent contact between the ultrasound emission element and a wall of a blood vessel or cavity. Optionally, the extension is transparent to ultrasound energy emitted by the ultrasound emission element.

There is provided in accordance with an exemplary embodiment of the invention an apparatus for keeping an ultrasound emission element away from a wall of a blood vessel or cavity comprising: a catheter comprising an ultrasound emission element; and a sheath, the catheter guided inside sheath into a blood vessel or a lumen, and wherein the sheath is configured to prevent contact between the ultrasound emission element and a wall of a blood vessel or cavity.

There is provided in accordance with an exemplary embodiment of the invention an apparatus for keeping an ultrasound emission element away from a wall of a blood vessel or cavity comprising: a deformable catheter comprising an ultrasound emission element, wherein the deformation is configured to prevent contact between the ultrasound emission element and a wall of a blood vessel or cavity. Optionally, the deformation comprises deforming between a first state, the first state comprises preventing contact between the ultrasound emission element and the wall, and a second state, the second state comprises allowing contact between the ultrasound emission element and the wall.

There is provided in accordance with an exemplary embodiment of the invention a catheter for medical therapy comprising: a distal end; and a proximal end comprising: an ultrasound emission element comprising an acoustic element; and one or more wires, at least one end of the one or more wires coupled at least proximal or distal to the element, the one or more wires forming a substantially looping shape, and a dimension of the looping shape sufficient to prevent contact between the acoustic element and a vessel wall.

In an exemplary embodiment of the invention, the one or more wires are arranged along the long axis of the catheter. Alternatively or additionally, the one or more wires are arranged perpendicular to the long axis of the catheter. Alternatively or additionally, the one or more wires are arranged along the circumference of the catheter.

In an exemplary embodiment of the invention, a first end of the one or more wires is coupled to the catheter and a second end of the one or more wires is not coupled to the catheter.

In an exemplary embodiment of the invention, the dimension of the looping shape is adjustable.

In an exemplary embodiment of the invention, the dimension of the one or more wires is sufficiently large to anchor the catheter inside a blood vessel.

In an exemplary embodiment of the invention, the one or more wires are not substantially within an ultrasound beam produced by the element.

In an exemplary embodiment of the invention, the one or more wires do not substantially block a flow of blood over the acoustic element.

In an exemplary embodiment of the invention, a diameter of the one or more wires is between 30 micrometers and 200 micrometers.

There is provided in accordance with an exemplary embodiment of the invention a catheter for medical therapy comprising: a distal end; and a proximal end comprising: an ultrasound emission element comprising an acoustic element; and one or more coils, at least one end of the one or more coils coupled at least proximal or distal to the element, and a dimension of the one or more coils sufficient to prevent contact between the acoustic element and a vessel wall.

In an exemplary embodiment of the invention, the proximal end of the catheter is positioned along a long axis of the one or more coils. Optionally, the one or more coils are a spiral. Optionally, the one or more coils are positioned around an outer circumference of the proximal end of the catheter. Optionally or additionally, the one or more coils comprises one or more springs. Optionally or additionally, the one or more spring comprises at least two regions having at least two spring constants.

In an exemplary embodiment of the invention, the wires are made of nitinol.

In an exemplary embodiment of the invention, the wires have a shape memory in a way that is bent when in a guiding catheter but reshaped to an original shape in the vessel.

In an exemplary embodiment of the invention, the catheter further comprising an open or close mechanism. Optionally, the mechanism comprises a wire that mechanically opens or closes the wire by pulling or pushing the wire. Optionally, the mechanism comprises a sheath that mechanically opens or closes the wire by covering or uncovering the memory shaped wires.

There is provided in accordance with an exemplary embodiment of the invention a catheter for medical therapy comprising: a distal end; and a proximal end comprising: an ultrasound emission element comprising an acoustic element; and one or more sheets, at least one edge of the one or more sheets coupled at least proximal or distal to the element, the sheets expand sufficiently to prevent contact between the acoustic element and a vessel wall.

In an exemplary embodiment of the invention, the catheter further comprises an open or close mechanism. Optionally, the mechanism comprises a wire that mechanically opens or closes the sheet. Optionally or additionally, the mechanism comprises one or more opening stages, the stage adapted to a diameter of the vessel.

There is provided in accordance with an exemplary embodiment of the invention a catheter for medical therapy comprising: a distal end; and a proximal end comprising: an ultrasound emission element comprising an acoustic element; and the proximal end of the catheter adapted to a substantially ellipsoid shape, and a dimension of ellipsoid sufficient to prevent contact between the acoustic element and a vessel wall.

There is provided in accordance with an exemplary embodiment of the invention a catheter for medical therapy comprising: a distal end; and a proximal end comprising: an ultrasound emission element comprising an acoustic element; a device to anchor the proximal end inside a blood vessel, the device comprising an axle, the axle adapted to provide rotation of the catheter without rotation of the device and the device prevents contact between the acoustic element and a wall of the vessel.

There is provided in accordance with an exemplary embodiment of the invention to a catheter for medical therapy comprising: a distal end; and a proximal end comprising: an ultrasound emission element comprising an acoustic element; one or more wires, at least one end of the one or more wires coupled at least proximal or distal to the element, the one or more wires forming a substantially looping shape, and a dimension of the looping shape sufficient to prevent contact between the acoustic element and a vessel wall; and a modification element coupled to the one or more wires, the modification element having a spacing of about half a wavelength of a frequency of an ultrasound beam, and the modification element configured to at least one of spread and filter the ultrasound beam.

In an exemplary embodiment of the invention, the modification element is positioned over at least some surface of the ultrasound emission element. Optionally, the modification element comprises a lattice. Alternatively or additionally, the modification element comprises a coil.

There is provided in accordance with an exemplary embodiment of the invention a method of using a guiding sheath to repositioning an ultrasound catheter coupled to a separation element comprising: deforming a separation element coupled to a catheter to a space between a guiding sheath and the catheter; repositioning the catheter; and reforming the separation element.

There is provided in accordance with an exemplary embodiment of the invention a method of using a retractable separation element to repositioning an ultrasound catheter comprising: retracting a separation element; repositioning the catheter; and reforming the separation element.

There is provided in accordance with an exemplary embodiment of the invention a catheter for medical therapy comprising: a distal end; and a proximal end comprising: an ultrasound emission element comprising an acoustic element; and an braiding cylinder from the proximal end, the cylinder sufficient to prevent contact between the acoustic element and a vessel wall.

There is provided in accordance with an exemplary embodiment of the invention a catheter for medical therapy comprising: a distal end; and a proximal end comprising: an ultrasound emission element comprising an acoustic element; and a balloon, the balloon having a volume sufficient to prevent contact between the acoustic element and to a vessel wall without blocking blood flow between the ultrasound emission element and the vessel wall.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storage instructions and/or data and/or a non-volatile storage, for example, a magnetic hard-disk and/or removable media, for storage instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for to purposes of illustrative discussion of embodiments of the invention. In this regard, the description taken with the drawings makes apparent to those skilled in the art how embodiments of the invention may be practiced.

In the drawings:

FIG. 1A is an illustration of an exemplary catheter for ultrasound treatment, in accordance with an exemplary embodiment of the invention;

FIG. 1B is a flow chart of an exemplary treatment method, in accordance with an exemplary embodiment of the invention;

FIG. 1C is a block diagram of an exemplary control system, in accordance with an exemplary embodiment of the invention;

FIGS. 2A-B illustrate an exemplary separation element coupled to a catheter, in accordance with an exemplary embodiment of the invention;

FIGS. 3A-C illustrate the use of arcs as separation elements to prevent and/or reduce contact with a vessel wall, useful in practicing an exemplary embodiment of the invention;

FIGS. 4A-D are images and illustrations of experimental results illustrating the effects of ultrasound treatment on tissue with and without the use of the separation element, useful in practicing an exemplary embodiment of the invention;

FIGS. 5A-B illustrate the use of the separation element to anchor and/or position the catheter inside the blood vessel, in accordance with an exemplary embodiment of the invention;

FIGS. 5C-D illustrate anchoring a proximal end of the catheter, in accordance with an exemplary embodiment of the invention;

FIGS. 5E-F illustrate anchoring using one or more springs and/or coils, in accordance with some embodiments of the invention;

FIG. 6 is a flow chart of a method of using the guiding sheath to reposition and/or reorient the catheter, in accordance with an exemplary embodiment of the invention;

FIGS. 7A-C illustrate a method of FIG. 6 in using a guiding sheath to reposition and/or reorient the catheter, useful in practicing an exemplary embodiment of the invention;

FIG. 7D illustrates the use of sheath 1704 as a separation device, in accordance with some embodiments of the invention;

FIG. 8 is a flow chart of a method of using the retractable separation element to reposition and/or reorient the catheter, in accordance with some embodiments of the invention;

FIGS. 9A-D illustrate the method of FIG. 8 in using a retractable separation element to reposition and/or reorient the catheter, useful in practicing some embodiments of the invention;

FIG. 10A illustrates a retractable separation element coupled to guidewire, in accordance with some embodiments of the invention;

FIGS. 10B-C illustrate the use of at least one longitudinal arc as the separation element, in accordance with some embodiments of the invention;

FIG. 11A illustrates an embodiment of the separation element designed to reduce the force applied to the wall, in accordance with some embodiments of the invention;

FIG. 11B illustrates the separation element of FIG. 11A contacting the wall, useful in practicing some embodiments of the invention;

FIGS. 12A-B illustrate an adjustable distance, in accordance with some embodiments of the invention;

FIGS. 13A-B illustrate the separation element deployed in situ, in accordance with some embodiments of the invention;

FIG. 14 illustrates a separation element that provides for rotation of the acoustic element, in accordance with some embodiments of the invention;

FIGS. 15A-D illustrate some non-limiting designs of the separation element, in accordance with some embodiments of the invention;

FIG. 16 is a flowchart of a method of calibration, in accordance with an exemplary embodiment of the invention;

FIG. 17A illustrates an element designed to modify an ultrasound beam, in accordance with an exemplary embodiment of the invention;

FIGS. 17B-C illustrate a a non-limiting example of the modification element, in accordance with an exemplary embodiment of the invention;

FIG. 17D illustrates an exemplary design of the modification element to modify an ultrasound beam to have a substantially uniform ultrasonic field, in accordance with some embodiments of the invention;

FIGS. 18A-B are illustrations of the modification element used as a separation element, in accordance with an exemplary embodiment of the invention; and

FIG. 18C illustrates an exemplary coil design of the separation element, in accordance with some embodiments of the invention.

### DESCRIPTION OF SPECIFIC EMBODIMENTS OF THE INVENTION

The present invention, in some embodiments thereof, relates to an ultrasound treatment system and, more particularly, but not exclusively, to a device for maintaining at least a distance between an ultrasound emission element and a tissue.

An aspect of some embodiments of the invention relates to a separation device to maintain a distance between an acoustic emission element (e.g., a transducer or a transmitter) on a catheter and a tissue, non-limiting examples of tissue include a wall of a blood vessel and/or cavity. In an exemplary embodiment of the invention, the separation device provides for at least some volume of blood flow over the element, for example, between the element and the wall.

In an exemplary embodiment of the invention, the distance is set to be constant. Optionally or alternatively, the distance is controllable and/or adjustable.

In an exemplary embodiment of the invention, the device is used to anchor the catheter inside the vessel.

In an exemplary embodiment of the invention, the separation device prevents and/or reduces damage to the element and/or to the wall. Optionally, at least some of the contact force between the device and the wall is absorbed and/or transferred. Alternatively or additionally, movement of the device against the wall (e.g., scraping endothelium) is reduced and/or prevented.

In an exemplary embodiment of the invention, the separation device is positioned to not interfere with the ultrasound beam produced by the transducer. Alternatively, the device interferes relatively with the ultrasound beam, for example, relatively little interference such as by interfering with the edges of the beam.

In an exemplary embodiment of the invention, the separation device is made out of a relatively thin material such as a wire. Optionally, at least one end is coupled to the to catheter relative to the ultrasound transducer, for example, proximal, distal, and/or on a side of the transducer. Optionally, the separation device is shaped like an arc. Alternatively or additionally, the separation device is shaped like a coil and/or spring.

In an exemplary embodiment of the invention, there is at least one separation device, optionally more than one, for example, 2, 4, 10, or other smaller, intermediate or larger numbers of devices.

In an exemplary embodiment of the invention, the separation device is made out of a relatively flexible and/or elastic material. Optionally, the material has memory.

An aspect of some embodiments of the invention relates to repositioning and/or reorienting a catheter with an ultrasound transducer inside the blood vessel without causing damage (e.g., reducing the risk of damage) to the blood vessel wall.

Optionally, the separation device is coupled to the catheter using at least one axle.

In an exemplary embodiment of the invention, the separation device conforms to a space between a catheter and a guiding sheath. Optionally the guiding sheath can be used to expand and/or retract the separation device.

In an exemplary embodiment of the invention, the separation device is expandable and/or retractable. Optionally, the separation device can be coupled to and/or uncoupled from (e.g., insertable and/or removable) the catheter.

In an exemplary embodiment of the invention, the separation device is collapsible during insertion and/or removal.

In an exemplary embodiment of the invention, the separation device prevents rotation of the transducer and/or catheter, for example, by anchoring the transducer and/or catheter to the vessel wall. Alternatively or additionally, the amount of rotation of the catheter and/or transducer is controllable by using the separation device. Alternatively or additionally, the separation device can be used to angle the transducer relative to the vessel wall.

In an exemplary embodiment of the invention, the separation device is used to control the flow rate of a liquid (e.g., blood) between the acoustic element and the vessel wall, for example, by relatively increasing and/or decreasing the distance between the acoustic element and the wall.

An aspect of some embodiments of the invention relates to an element designed to modify at least some portion of an ultrasound beam produced by a transducer.

Optionally, the modification element comprises at least some portion of a separation device.

In an exemplary embodiment of the invention, the modification element is a lattice. Alternatively or additionally, the modification element is a coil.

In an exemplary embodiment of the invention, the modification element spreads out the ultrasound beam. Alternatively or additionally, the element filters the beam, for example, the first harmonic is filtered out.

An aspect of some embodiments of the invention relates to a method for keeping an ultrasound transducer away from a wall of a blood vessel or cavity comprising inserting a catheter comprising a transducer into a blood vessel or body cavity such that said transducer is prevented from contacting a wall of said blood vessel or said cavity. Optionally, a minimum distance between the transducer and the wall is maintained. Alternatively or additionally, the distance is constant. Alternatively, the distance is variable. Optionally, the distance is controllable and/or settable.

An aspect of some embodiments of the invention relates to an apparatus for keeping an ultrasound transducer away from a wall of a blood vessel or cavity comprising an extension on a catheter comprising an ultrasound transducer, said extension configured to prevent contact between said transducer and a wall of a blood vessel or cavity. Optionally, said extension is transparent to ultrasound energy emitted by said ultrasound transducer.

An aspect of some embodiments of the invention relates to an apparatus for keeping an ultrasound transducer away from a wall of a blood vessel or cavity comprising a catheter comprising an ultrasound transducer; and a sheath, said catheter guided inside sheath into a blood vessel or a lumen, and wherein said sheath is configured to prevent contact between said transducer and a wall of a blood vessel or cavity.

An aspect of some embodiments of the invention relates to an apparatus for keeping an ultrasound transducer away from a wall of a blood vessel or cavity comprising: a deformable catheter comprising an ultrasound transducer, wherein said deformation is configured to prevent contact between said transducer and a wall of a blood vessel or cavity. Optionally, the catheter deforms between a first state of to preventing contact between the transducer and the wall, and a second state of allowing contact between the transducer and the wall.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Overview

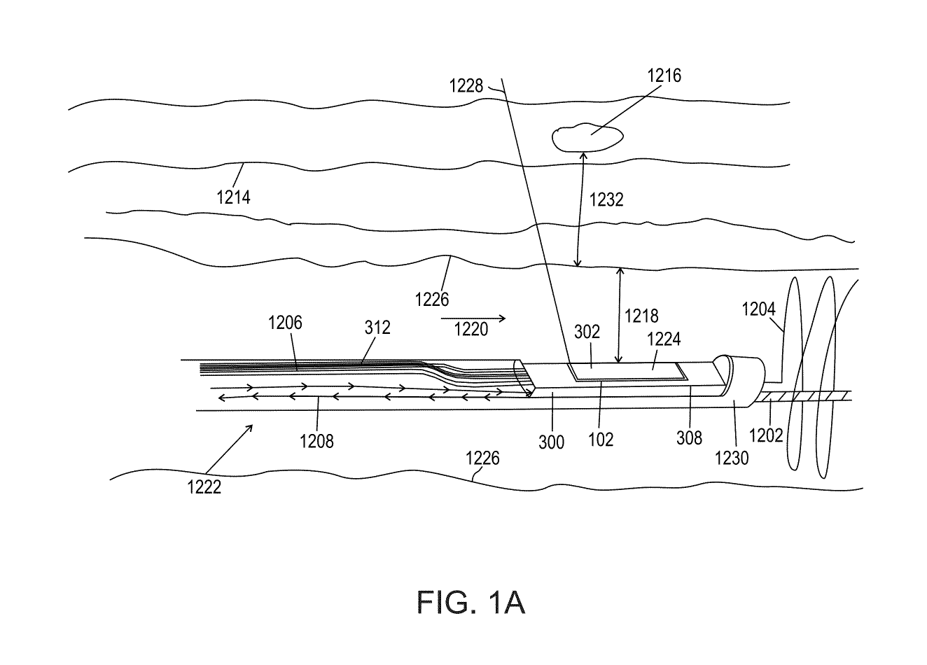

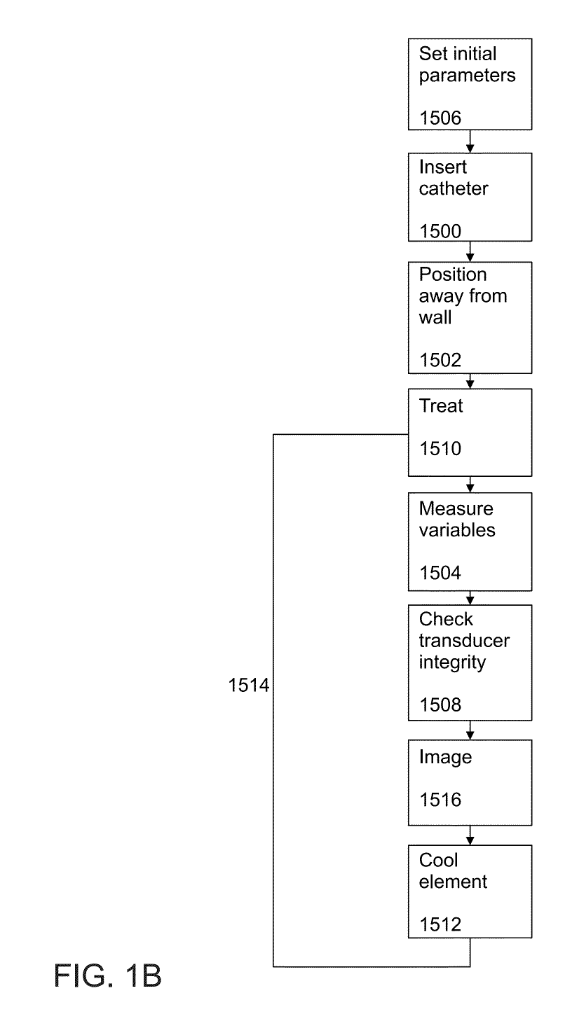

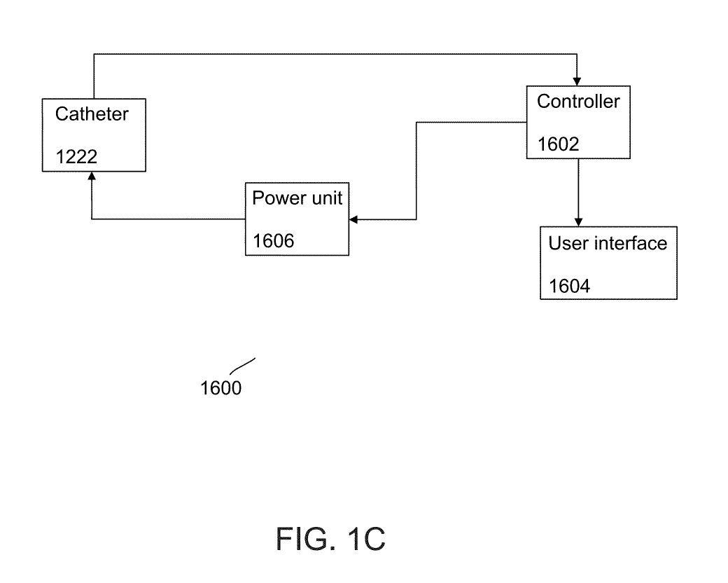

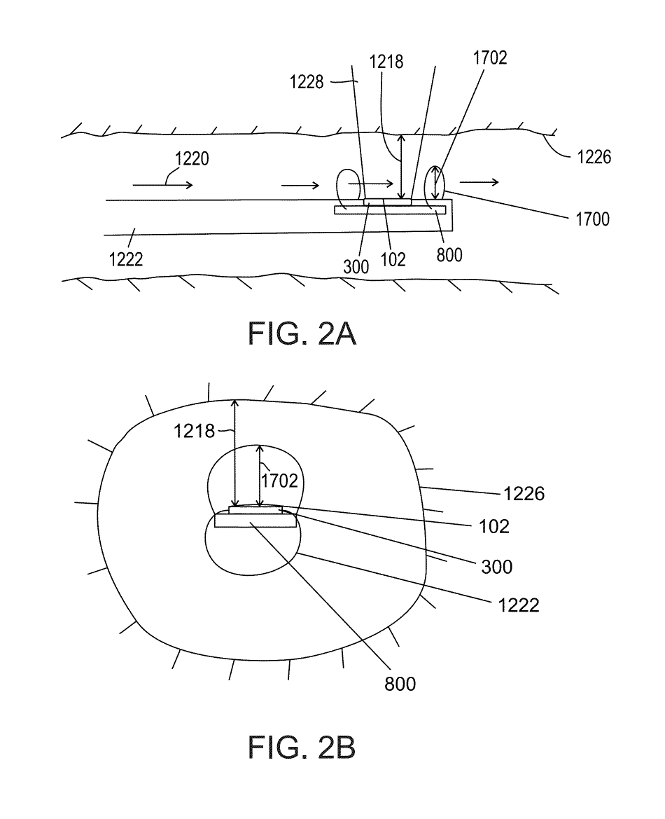

For purposes of better understanding some embodiments of the invention, as illustrated in FIGS. 2-18 of the drawings, reference is first made to an example of an ultrasound treatment catheter 1222 inside a blood vessel, as illustrated in FIG. 1A, and/or a method of treatment, as illustrated in FIG. 1B, where exemplary embodiments of the invention may be used.

At 1506, one or more initial parameters are set (e.g., for treatment, for imaging) in accordance with an exemplary embodiment of the invention, for example, frequency, energy intensity, duty cycle, pulse duration, pulse repetition frequency, duration of treatment, focused and/or unfocused mode, maximum temperature for element 102. In an exemplary embodiment of the invention, the initial parameters are set according to a treatment plan, for example, as described with reference to co-filed PCT application “Tissue Treatment” (attorney docket number 52347), incorporated herein by reference in its entirety. Optionally, the treatment plan is based on clear anatomical landmarks, such as arterial bifurcations.

At 1500, catheter 1222 in inserted into the body of a patient, in accordance with an exemplary embodiment of the invention. Standard vascular access methods can be used, for example, from the femoral artery. Optionally, catheter 1222 is threaded using a guide wire 1202 (e.g., over the wire, rapid exchange) to the target treatment site (e.g., an artery such as the iliac, renal, carotid, aorta) under image guidance, such as fluoroscopy. Alternatively or additionally, catheter 1222 is directed inside a guiding sheath to the anatomical treatment location.

In an exemplary embodiment of the invention, the initial parameters are set manually (e.g., by a user) using an interface coupled to a controller. Alternatively or additionally, parameters are automatically determined, such as by a software module of controller. Additional details about the controller will be provided with reference to FIG. 1C.

In an exemplary embodiment of the invention, catheter 1222 comprises at least one transducer 300, positioned for example, on the side, such as inside a window cut into the catheter shaft 1230. Alternatively, a support for transducer 300 is “U” shaped.

At 1502, contact between an acoustic element 102 of transducer 300 and a surface and/or a wall 1226 of a vessel, cavity and/or lumen is reduced and/or prevented, for example, by a separation element and/or device 1204, in accordance with an exemplary embodiment of the invention. Device 1204 maintains a distance 1218 between element 102 and wall 1226 of at least 1 mm. In an exemplary embodiment of the invention, maintaining at least distance 1218 reduces or prevents overheating of element 102. Optionally, a fluid located in distance 1218 transfers heat away from element 102.

At 1510, electrical energy is applied to transducer 300, for example, according to parameters set at 1506, in accordance with an exemplary embodiment of the invention.

In an exemplary embodiment of the invention, target tissue 1216 is treated by an ultrasound beam 1228 from transducer 300. In some embodiments, treating comprises a thermal effect (e.g., heating to above 55 degrees Celsius) and/or a cavitation effect. In some embodiments, damage and/or treatment to tissues (e.g., normal, healthy) surrounding target tissue 1216 is reduced and/or prevented. In some embodiments, treatment and/or damage to a volume of tissue between target tissue 1216 and wall 1226 is reduced and/or prevented. Selection of tissue is discussed in more detail with reference to PCT application “Tissue Treatment” (attorney docket number 52347).

Optionally, at 1504, one or more variables are measured and/or estimated as part of treatment feedback, for example, a flow rate of blood 1220 inside the vessel (e.g., using transducer 300 in Doppler mode), a temperature of transducer 300 (e.g., using sensor 308), distance 1218 (e.g., using acoustic feedback), distance 1232 (e.g., using transducer 300 in imaging mode), and/or impedance of transducer 300 (e.g., electrical measurements), in accordance with an exemplary embodiment of the invention. Distances 1218 and/or 1232 can be measured, for example, as described with reference to “Ultrasound Transceiver and Uses in Detection” (attorney docket no. 52342). Other measurements and/or estimates taught by application 52342 can also be performed, for example, detection of pulsations, and/or the diameter of the artery. Optionally or alternatively, an ultrasonic distance measuring element is used.

In some embodiments, variables measured at 1504 are used to calibrate and/or adjust parameters at 1506, for example, by a look-up table of correlated values. Optionally, measurements as in 1504 occur during and/or after the treatment. Optionally or additionally, adjustment of parameters as in 1506 occurs during and/or after the treatment.

Optionally, at 1508, the integrity of transducer 300 is verified, for example, for mechanical failure and/or presence of foreign matter (e.g., thrombus), in accordance with an exemplary embodiment of the invention. Integrity is verified, for example, by measuring changes in the impedance of transducer 300. Optionally, verification of integrity occurs during and/or after treatment.

Measuring the integrity of transducer 300 is described with more detail with reference to co-filed PCT application “An Ultrasound Transceiver and Uses Thereof” (attorney docket number 52345), incorporated herein by reference in its entirety.

Optionally, at 1516, transducer 300 is used in an imaging mode to obtain feedback about a target tissue 1216, such as by receiving ultrasound energy echoes from the tissues. One or more non-limiting examples of target tissues 1216 include, fat, nerves, vasa vasora, lymph, tumor, connective tissue, plaque (e.g., atherosclerotic). Target tissue 1216 may be located a distance 1232 away from the inner surface of wall 1226. Examples of the maximum distance 1232 of target tissue 1216 that can be imaged using transducer 300 include 0.5 mm, 1 mm, 2 mm, 5 mm, 10 mm, or other smaller, intermediate or larger distances. Alternatively or additionally, one or more non-limiting examples of ultrasound imaging methods to estimate the extent of thermal damage in the target tissue and/or surrounding tissue include, measuring the ultrasound backscatter coefficient, ultrasound elastography, measuring US absorption and/or scattering from the treatment region, spectral signature mapping, classification according to a classification matrix of tissues, and the ultrasonic effect.

In some embodiments, element 102 is used to receive ultrasound energy, for example, returning echoes, such as during imaging of tissues. Receiving ultrasound energy can create a voltage across electrodes 302 and/or 304. Optionally, emission element 102 can function both as an emitter and receiver, for example, as a transceiver. Emission element 102 and/or a catheter may be provided with an acoustic/ultrasonic transducer.

In an exemplary embodiment of the invention, ultrasound emission element 102 is an unfocused emission element. For example, the beam produced by element 102 does not focus and/or converge at a point. For example, the beam produced by element 102 stays substantially straight and/or slightly diverges (e.g., about 15 degrees) after leaving element 102. Optionally, element 102 is a widebeam emission element, for example, the beam produced by element 102 diverges more than about 15 degrees after leaving element 102.

Optionally, at 1512, element 102 is cooled, in accordance with an exemplary embodiment of the invention. Optionally, cooling occurs by transfer of heat from element 102 to a surrounding fluid such as blood 1220, saline, urine, water, angiography contrast fluids, cerebrospinal fluid, lymph, mucous, stomach acid. Alternatively or additionally, cooling occurs by injection of a cooled volume of a liquid (e.g., saline, radio-opaque dye) through tube 1206, and/or circulation of a liquid through tube 1208. Alternatively or additionally, cooling is increased using active heat flux, such as the thermoelectric cooler.

In some embodiments, cooling of element 102 is controlled by the controller, using feedback obtained about the temperature of element 102, for example, from sensor 308.

Additional details about cooling element 102 are discussed with reference to co-filed PCT application “An Ultrasound Transceiver and Cooling Thereof” (attorney docket number 52346), incorporated herein by reference in its entirety.

Optionally, at 1514, one or more of 1504, 1516, 1508, 1510 and/or 1512 are repeated, for example, in a feedback cycle.

### Exemplary Control System

FIG. 1C illustrates an exemplary ultrasound treatment system 1600 for selectively treating tissues, in accordance with an exemplary embodiment of the invention. System 1600 provides for the control of the ultrasound treatment and/or monitoring of the treatment using catheter 1222. A transducer 300 comprising an acoustic element 102 to produce ultrasound energy is optionally located on a distal end of catheter 1222.

In an exemplary embodiment of the invention, an operator (e.g., physician performing the procedure) programs a controller 1602 (e.g., computer) for treatment using a user interface 1604 (e.g., keyboard, mouse, monitor). Optionally, treatment is monitored, for example, by viewing feedback parameters on interface 1604.

In an exemplary embodiment of the invention, a power port unit 1606 provides voltage and/or current (e.g., alternating and/or oscillating) electrical power to electrodes across element 102, causing element 102 to vibrate (e.g., expand and/or contract) at the set frequency, outputting a set ultrasound intensity profile.

In an exemplary embodiment of the invention, one or more functions and/or parameters and/or settings are programmed and/or set into controller 1602 (e.g., automatically determined by software such as according to a treatment plan). Optionally or additionally, one or more functions and/or parameters are selectable (e.g., manually set by user, automatically selected by software).

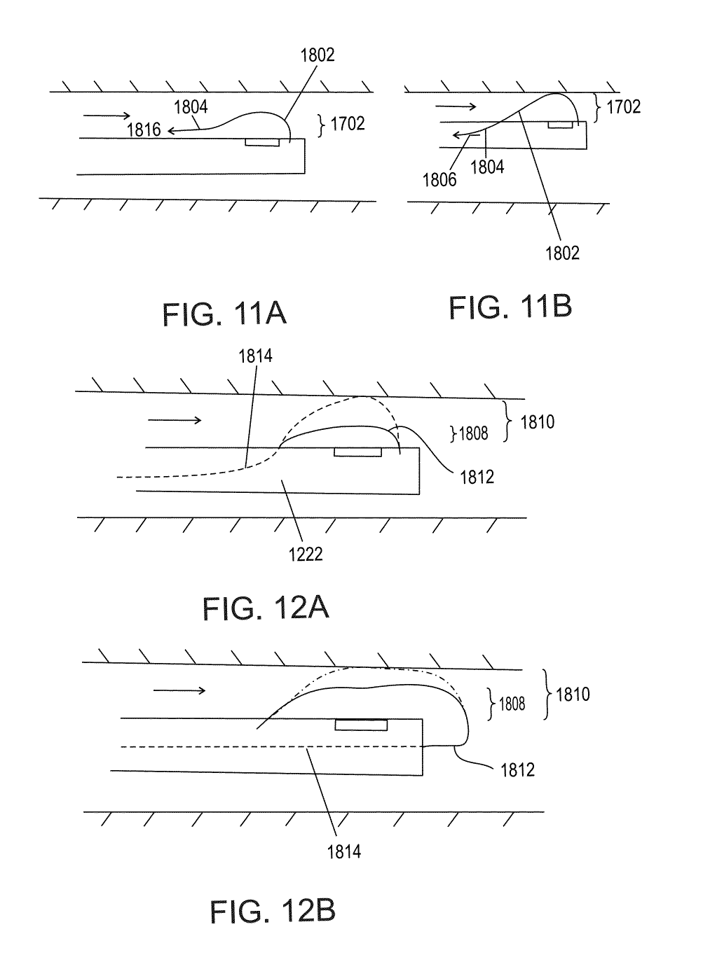

One or more non-limiting examples of settable parameters include, for example:
Impedance of element 102.
Acoustic feedback is feedback obtained by analyzing returning echoes of a diagnostic ultrasound signal returning from tissues, for example, the voltage across element 102 as a function of time, for example, as will be described in more detail with reference to FIG. 11.
Estimated or measured flow rate of blood across the surface of the acoustic element is the estimated (e.g., average) rate of flow of fluid (e.g., blood) across the surface of element 102, important for controlling the temperature of the element to prevent overheating.
Estimated or measured flow rate of blood across and/or the wall of the treatment target (e.g., blood vessel) is important for estimating the cooling capacity of the blood on the tissues of the wall being heated by ultrasound.
Efficiency is the estimated efficiency of converting electrical energy into ultrasound energy by the acoustic element 102.
Cooling system cools element and/or blood vessel wall 102 to the desired temperature of operation. Optionally, the cooling system is used in combination with the blood flow.
Impulse excitation is the application of an impulse function (e.g., delta function) to the element 102, causing the element 102 to vibrate with decreasing amplitude Impulse excitation is used to estimate a reduction in efficiency, useful as feedback, for example, to determine one or more of, thrombus formation on the element, the element coming in contact with the vessel wall, mechanical damage to the element.
Navigation system controls the movement and/or positioning and/or orientation of catheter 1222 and/or the transducer 300.
Pressure is the pressure caused by sound (e.g., acoustic intensity) of the liquid (e.g., blood) during treatment and/or imaging.
Electric power is the applied power to the transducer.
Reflected electric power from the transducer back to the controller.
Voltage is the measured and/or applied voltage on the transducer.
Current is the measured and/or applied current in the transducer.

One or more non-limiting examples of selectable parameters include:
Frequency of the ultrasound energy produced by vibration of the acoustic element 102.
Waveform, applied to the acoustic element, for example, using a sinusoidal wave form.
Intensity is the produced ultrasound energy power divided by the surface area of the acoustic element.
Pulse duration is the length of a pulse of acoustic energy measured in time.
Duty cycle is the percentage of time in a single pulse that ultrasound energy is transmitted.
Temperature of operation threshold is the approximate temperature of the element 102 and/or the liquid (e.g., blood, saline) that should not be exceeded.
Treatment pattern is the spatial and/or temporal combination of one or more of the above variables, for example, a single pulse, a sequence of pulses, train of pulses.
Focusing is the setting of non-focused vs. focused ultrasound energy.

The table below sets out some examples of the selectable parameters, and provides their theoretical limits, an exemplary treatment range, and an exemplary treatment sub range (e.g., most commonly used settings). It is important to note that some selectable parameters can only be selected from a pre-determined set, for example, in some embodiments, catheters are designed to operate at a specific frequency, in which case the user selects the frequency according to the catheter available.

| | | Exemplary | Exemplary |
|---|---|---|---|
| | | Treatment | Treatment sub |
| Parameter | Theoretical range | range | range |
| Frequency (MHz): | | | |
| Treatment | 1-60 | 8-30 | 10-22 |
| Imaging | 1-60 | 10-60 | 10-25 |
| Intensity (Watts/sq cm) | 1-200 | 10-100 | 10-60 |
| Duty cycle (%) | 0.1-100 | 10-100 | 50-100 |
| Pulse duration (seconds) | 0.01-1000 | 0.1-4 | 0.1-2 |
| Duration of treatment | 0.1-1000 | 2-120 | 3-60 |
| (Seconds) per location | | | |
| Efficiency (%) | 1-70% | 20-70% | 35-70% |
| Temperature (Celsius) | 10-100 | 15-80 | 25-80 |

### Separation Element—Exemplary Design

FIGS. 2A (side view) and 2B (cross sectional view) illustrate an exemplary design of a catheter 1222 comprising an ultrasound transducer 300, wherein catheter 1222 is coupled to a separation element, such as an arc 1700, in accordance with an exemplary embodiment of the invention. In an exemplary embodiment of the invention, arc 1700 prevents and/or reduces contact between the acoustic element (e.g., element 102) of transducer 300 and the wall (e.g., blood vessel wall 1226). Optionally, at least a distance 1702 is maintained between element 102 and wall 1226.

In an exemplary embodiment of the invention, distance 1702 is at least 0.1 mm, at least 1 mm, at least 2 mm, at least 5 mm, at least 10 mm, at least 30 mm, or other smaller, intermediate or larger distances are used.

In an exemplary embodiment of the invention, distance 1702 is less than and/or about equal to an actual distance 1218 from the nearest area of element 102 to wall 1226. For example, if arcs 1700 are not in contact with wall 1226 as shown in FIG. 2A, distance 1218 is greater than 1702.

In an exemplary embodiment of the invention, two arcs 1700 are used, for example, one arc 1700 positioned proximal to element 102 and one arc 1700 positioned distal to element 102. Optionally, arcs 1700 are perpendicular relative to the long axis of element 102 and/or catheter 1222.

In an exemplary embodiment of the invention, distance 1702 is determined by the dimensions of arc 1700, for example, the length along the diameter of arc 1700 to the surface of catheter 1222 and/or element 102. Optionally, the length from at least some areas of arc 1700 to element 102 is at least the value of distance 1702. Alternatively, the length from all areas of arc 1700 to element 102 is at least the value of distance 1702.

In an exemplary embodiment of the invention, arcs 1700 are made out of a material that is relatively flexible. Optionally, the material is biocompatible. Alternatively or additionally, the material is relatively elastic. Alternatively or additionally, the material has memory (e.g., returns to preset configuration after deformation and/or release). One or more non-limiting examples of the material include, a metal such as nitinol and/or stainless steel.

In an exemplary embodiment of the invention, at least some volume of a fluid, such as blood 1220, is present over the surface of element 102. Optionally, the fluid is present between element 102 and wall 1226.

In an exemplary embodiment of the invention, the diameter of the material of arcs 1700 (e.g., of the wire) is relatively small, for example, about 30 micrometers, about 50 micrometers, about 100 micrometers, about 200 micrometers, or other smaller, intermediate or larger diameters. Optionally, a flow rate of blood 1220 is relatively unchanged by the presence of one or more arcs 1700.

In an exemplary embodiment of the invention, arcs 1700 are positioned such that to they do not intersect an ultrasound beam 1228 produced by element 102. Alternatively or additionally, arcs 1700 intersect beam 1228, for example, interfering relatively little with beam 1228, for example, interfering with the edges of beam 1228.

In an exemplary embodiment of the invention, arcs 1700 are coupled to the shaft of catheter 1222, for example, by one or more non-limiting methods such as, welding, gluing, crimping. Optionally, arcs 1700 are coupled to a support structure of element 102 and/or transducer 300.

It should be noted that arc 1700 is used as a non-limiting term for the structure of the separation device, as other shapes can be used, for example, one or more of a rectangle, a triangle, a star.

In some embodiments of the invention, the acoustic energy outputted by the transducer is used as the separation device, the acoustic energy being sufficiently strong to prevent the transducer from touching the wall. Optionally, the acoustic energy is used in conjunction with an embodiment of the separation device as described herein.

Examples of Separation Element Protecting the Acoustic Element from Contacting the Vessel Wall

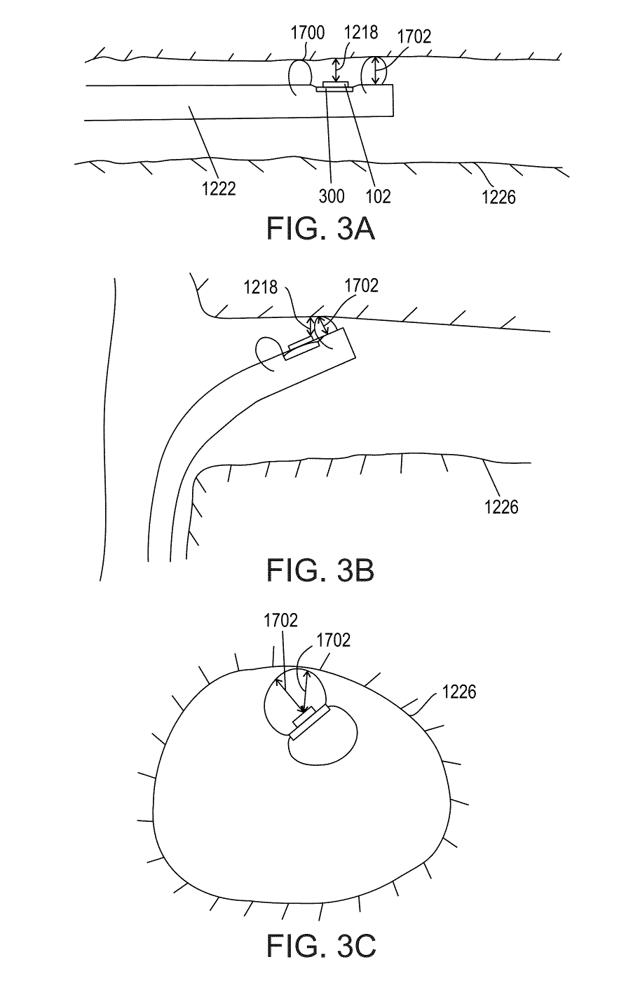

FIGS. 3A-C illustrate the use of arcs 1700 (e.g., catheter 1222 as shown in FIG. 2A) in preventing and/or reducing contact with wall 1226, for example, by acting as bumpers in contacting wall 1226, useful in practicing an exemplary embodiment of the invention. Optionally, a space between element 102 and wall 1226 is at least distance 1702.

In some embodiments, distance 1702 is substantially equal to distance 1218, for example, if arcs 1700 are in contact with wall 1226 (e.g., acting as bumpers) as in FIG. 3A.

As an example, as shown in FIG. 2A, arcs 1700 do not contact wall, such as if catheter 1222 is positioned inside a relatively large blood vessel.

Shown in FIG. 3A a side view of catheter 1222 having moved towards wall 1226 until being stopped by arcs 1700, for example, due to blood flow and/or manipulation by the physician

FIG. 3B illustrates a side view of catheter 1222 during a turn to enter a to branching vessel. Element 102 is prevented from contacting wall 1226 by arcs 1700.

In an exemplary embodiment of the invention, the separation device is used to align and/or angle the transducer relative to the vessel wall, for example, as shown in FIG. 3B. Optionally, arc 1700 placed distal to the transducer can be made relatively larger than arc 1700 placed proximal to the transducer (or vice versa) to provide for a set and/or controllable angle.

FIG. 3C illustrates a cross sectional view of catheter 1222 in a rotated position relative to wall 1226. Arcs 1700 prevent and/or reduce the risk of element 102 contacting wall 1226.

### Potential Advantages of Some Embodiments

A potential advantage of the separation device is preventing and/or reducing damage to element 102, for example, mechanical damage due to contact with wall 1226.

A potential advantage of the separation device providing for at least some blood (and/or other fluid) to be present over the surface of element 102, is preventing and/or reducing damage to element 102, such as due to an insufficient amount of heat transfer to the surrounding blood.

A potential advantage of the design of the separation device is reducing and/or preventing damage to wall 1226 such as a result of contact with element 102, one or more non-limiting examples include, triggering a coagulation cascade, causing a vessel spasm, causing stenosis, blood loss due to injury to the vessel wall.

A potential advantage of the separation design is controlling the distance between the element and the vessel wall to obtain a desired flow rate of blood. For example, increasing and/or decreasing the flow rate between the element and the wall in order to relatively increase and/or decrease the cooling rate of the acoustic element and/or vessel tissue.

### Experimental Results Showing Potential Advantages of Some Embodiments

Reference is now made to the following example, which together with the description herein illustrates some embodiments of the invention in a non-limiting fashion.

In an experiment using a living pig, inventors inserted a catheter with an ultrasound transducer into an artery using standard medical procedures. Inventors generated an ultrasonic intensity of approximately 40 Watt/square centimeter (non-limiting example of treatment intensity) at a frequency of about 10 Mhz (non-limiting example of treatment frequency) from the acoustic element to treat tissue in the wall of the blood vessel. When the acoustic element touched the arterial wall during excitation, the temperature around the element was estimated to be about 70 degrees Celsius.

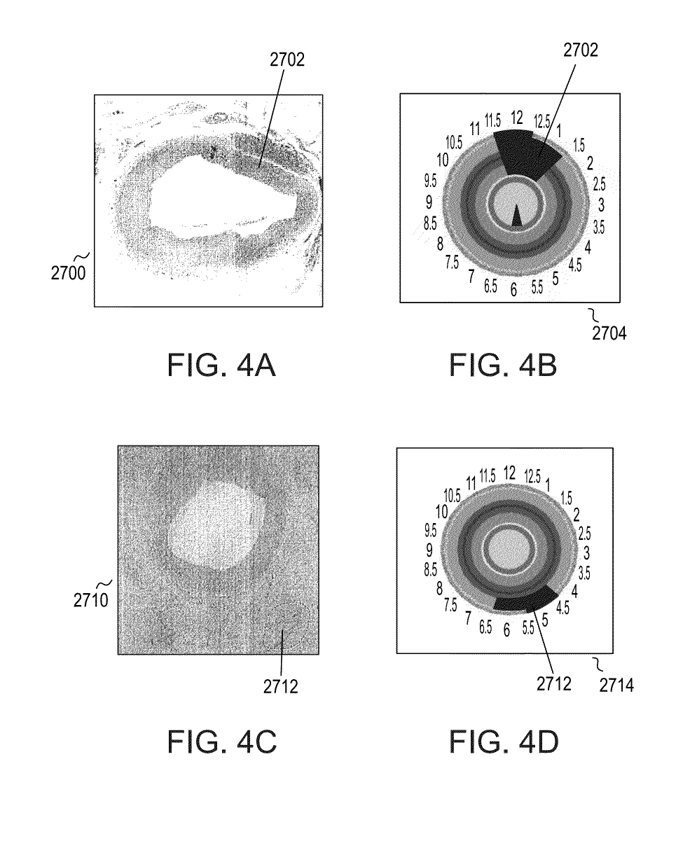

FIGS. 4A-D are images and/or illustrations of experimental results illustrating the effects of ultrasound treatment on tissue with and/or without the use of the separation element, useful in practicing an exemplary embodiment of the invention.

FIG. 4A is a histological slide of artery 2700. The contact between the acoustic element and the arterial wall is believed to have resulted in substantial thermal damage 2702 to the arterial wall, including damage to the endothelium.

FIG. 4B is a sketch illustration 2704 of artery 2700 to clarify the area of thermal damage 2702, shown as darkened regions.

For comparison purposes, in another experiment with a set-up similar to the experiment described above, inventors generated an ultrasonic output of approximately 26.5 Watt/cm² (non-limiting example of treatment intensity) at a frequency of about 11.3 Mhz (non-limiting example of treatment frequency). The temperature around the element was estimated at about 52.5 degrees Celsius. A distance (e.g., greater than zero) was maintained between the element and the arterial wall throughout treatment.

FIG. 4C is a histological slide of artery 2710. Maintaining the distance between the acoustic element and the arterial wall is believed to have resulted in targeted treatment, for example, treatment (e.g., thermal damage) to the peri-adventitia 2712, without causing damage to the endothelium.

FIG. 4D is a sketch illustration 2714 of artery 2710 to clarify the area of thermal damage to the peri-adventitia 2712, shown as darkened regions.

### Anchoring

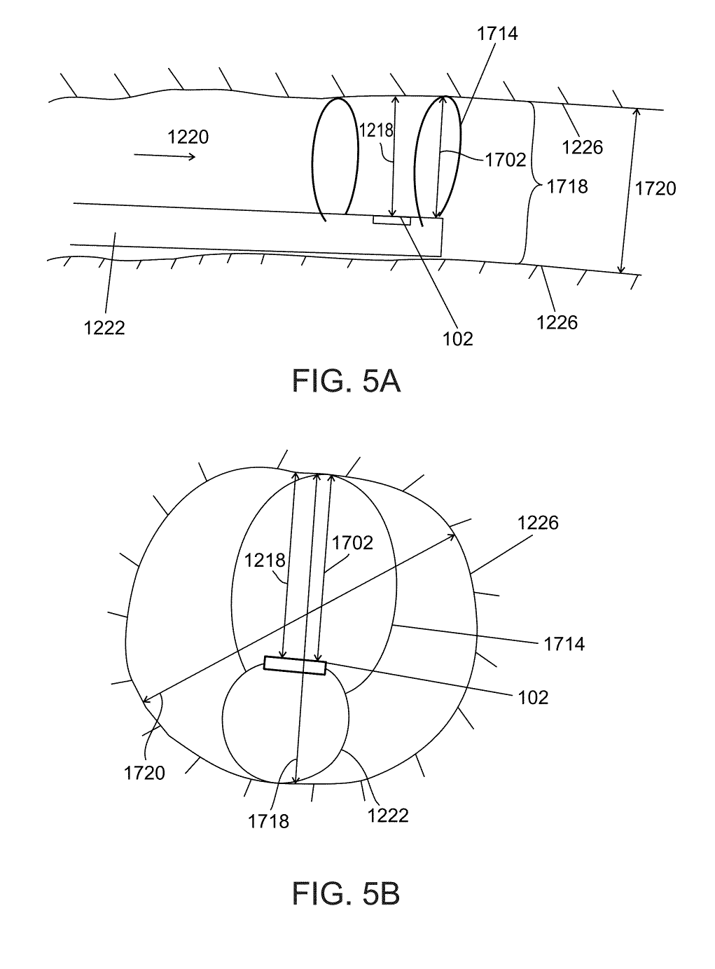

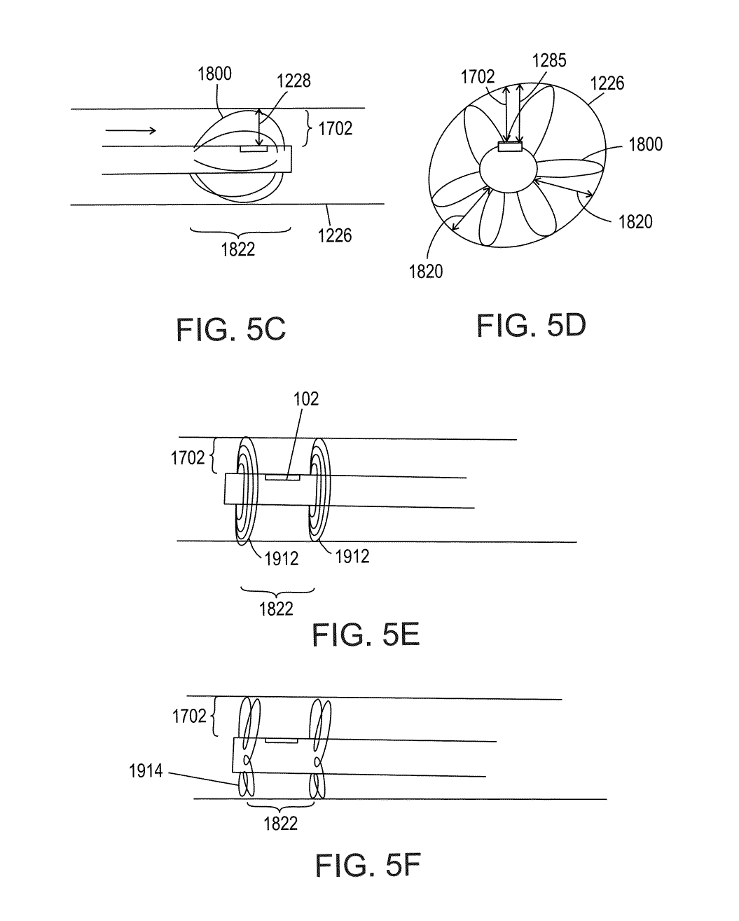

FIG. 5A is a side view and FIG. 5B is a cross sectional view of the separation device used to anchor and/or position the catheter of FIGS. 2A-B inside the blood vessel, such as against wall 1226, in accordance with an exemplary embodiment of the to invention. Optionally, the separation element is a relatively large arc 1714, similar to arc 1700. It should be noted that the term ‘relatively large’ is non-limiting, and in the context described herein refers to dimensions relative to the dimensions of the blood vessel.

In an exemplary embodiment of the invention, a cross sectional length 1718 from an end of large arc 1714 to an opposite end of catheter 1222 is approximately equal to a diameter 1720 of the blood vessel, for example, for a blood vessel diameter of 5 mm and catheter 1222 cross sectional diameter of 2 mm, the cross sectional diameter of arc 1714 (e.g., distance 1702) is about 3 mm or about 4 mm

FIGS. 5C-D illustrate an embodiment in which contact between a proximal end 1822 of catheter 1222 (e.g., containing element 102) is prevented and/or reduced between wall 1226 by anchoring, in accordance with an exemplary embodiment of the invention. Optionally, at least a gap 1820 (e.g., greater than zero) is maintained between proximal end 1822 and wall 1226.

In some embodiments, a plurality of arcs 1800, (e.g., arranged parallel to the long axis of catheter 1222), are distributed around the circumference (e.g., in cross section) of proximal end 1822. The number of arcs 1800 is non-limiting, for example, 4, 6, 8, or other smaller, intermediate or larger numbers of arcs 1800.

FIG. 5E illustrates an embodiment, wherein one or more relatively flat (e.g., spiral) springs and/or coils 1912 anchor proximal end 1822, for example, coils 1912 are placed proximally and/or distally relative to element 102, in accordance with some embodiments of the invention.

FIG. 5F illustrates an embodiment, wherein one or more helical shaped springs and/or coils 1914 anchor proximal end 1822, for example, oriented around the outer circumference of end 1822, in accordance with some embodiments of the invention.

A potential advantage of using the separation element to anchor and/or position catheter 1222 inside the blood vessel, is reducing and/or preventing damage to wall 1226. For example, position and/or orientation changes of catheter 1222 (e.g., to treat different parts of the artery) may result in scraping of catheter 1222 against the endothelium of wall 1226. A potential advantage of maintaining at least gap 1820 between proximal end 1822 and wall 1226 is reducing and/or preventing damage to wall to by contacting wall only with arcs 1800, potentially a gentler form of contact than proximal end 1822 of catheter 1222.

Exemplary Separation Element and Method of Use with a Sheath

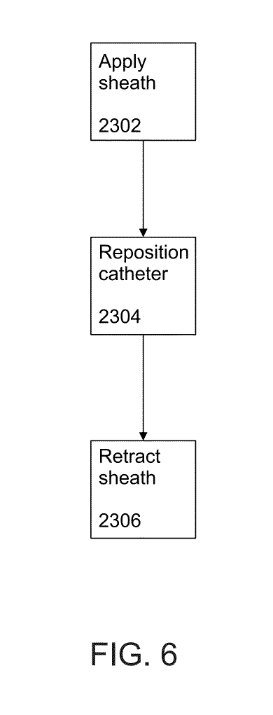

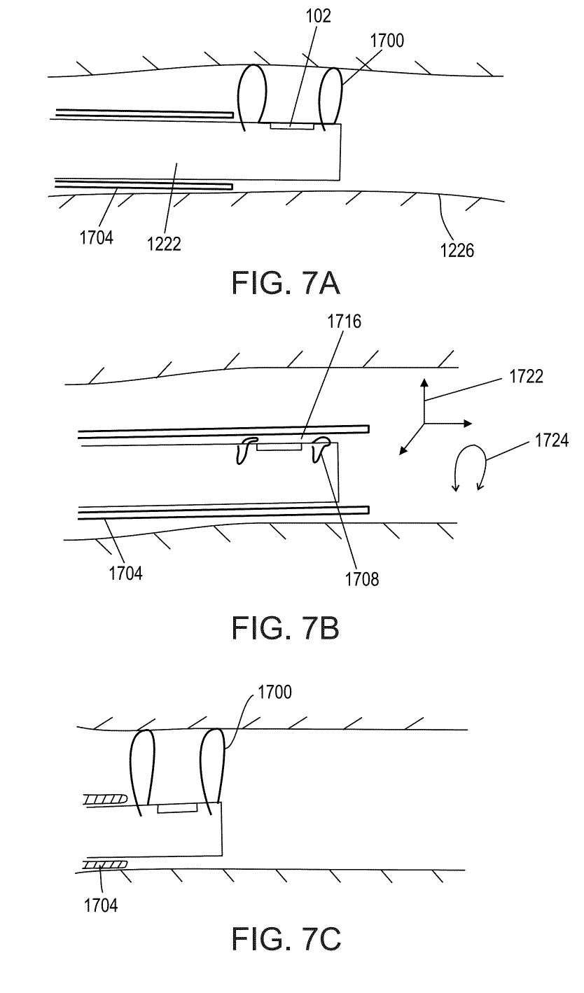

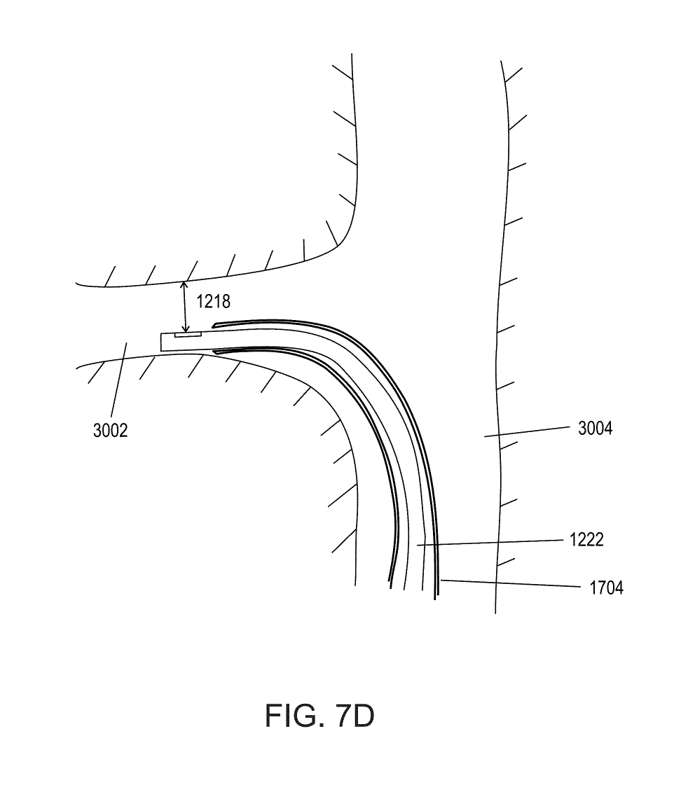

FIG. 6 is a method, illustrated by FIGS. 7A-C, of the use of a guiding sheath 1704 to reposition and/or re-orient catheter 1222, for example, during a medical procedure, in accordance with an exemplary embodiment of the invention.

FIG. 7A illustrates catheter 1222, anchored using the separation element (e.g., arc 1700) in the blood vessel, for example, as described with reference to FIGS. 5A-B, in accordance with an exemplary embodiment of the invention. Shown is catheter 1222 surrounded by sheath 1704. In some embodiments, catheter 1222 is not anchored, for example, as described with reference to FIGS. 2A-B.

At 2302, sheath 1704 is applied by proximal movement of sheath 1704 relative to catheter 1222, in accordance with an exemplary embodiment of the invention.

FIG. 7B illustrates catheter 1222 after sheath 1704 has been applied over arcs 1700. In an exemplary embodiment of the invention, conformed arcs 1708 represent arcs 1700 that have been deformed by sheath 1704 to a space 1716 between catheter 1222 and the internal surface of sheath 1704.

Optionally, at 2304, in accordance with an exemplary embodiment of the invention, catheter 1222 inside sheath 1704 is repositioned and/or reoriented, for example, displaced in space 1722 (e.g., up/down, forward/backwards, sideways) and/or rotated 1724 (e.g., clockwise/counterclockwise).

Optionally, at 2306, in accordance with an exemplary embodiment of the invention, sheath 1704 is retracted from catheter 1222, for example, by a distal movement relative to catheter 1222.

FIG. 7C illustrates catheter 1222 after retraction of sheath 1704 and/or distal repositioning of catheter 1222, for example, to treat another volume of tissue in the blood vessel wall 1226. In an exemplary embodiment of the invention, conformed arc 1708 returns to substantially the same configuration, for example arc 1700.

FIG. 7D illustrates the use of sheath 1704 as a separation device, in accordance to with some embodiments of the invention. In some embodiments, sheath 1704 is positioned away from the tissue wall, for example, sheath 1704 is placed inside a branch vessel 3002 (e.g., renal artery) off from a main vessel 3004 (e.g., aorta). Catheter 1222 is pushed out of the end of sheath 1704, exposing the transducer, placing transducer distance 1218 away from the wall of branch vessel 3002.

In some embodiments, sheath 1704 is designed to keep catheter 1222 away from the vessel wall, for example, sheath 1704 is designed to have a curvature (e.g., by selective molding of the plastic of sheath 1704) to match the curvature between main vessel 3004 and branch vessel 3002.

Exemplary Separation Element and Method of Use with a Wire

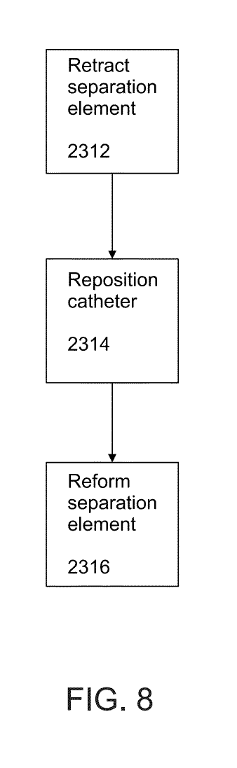

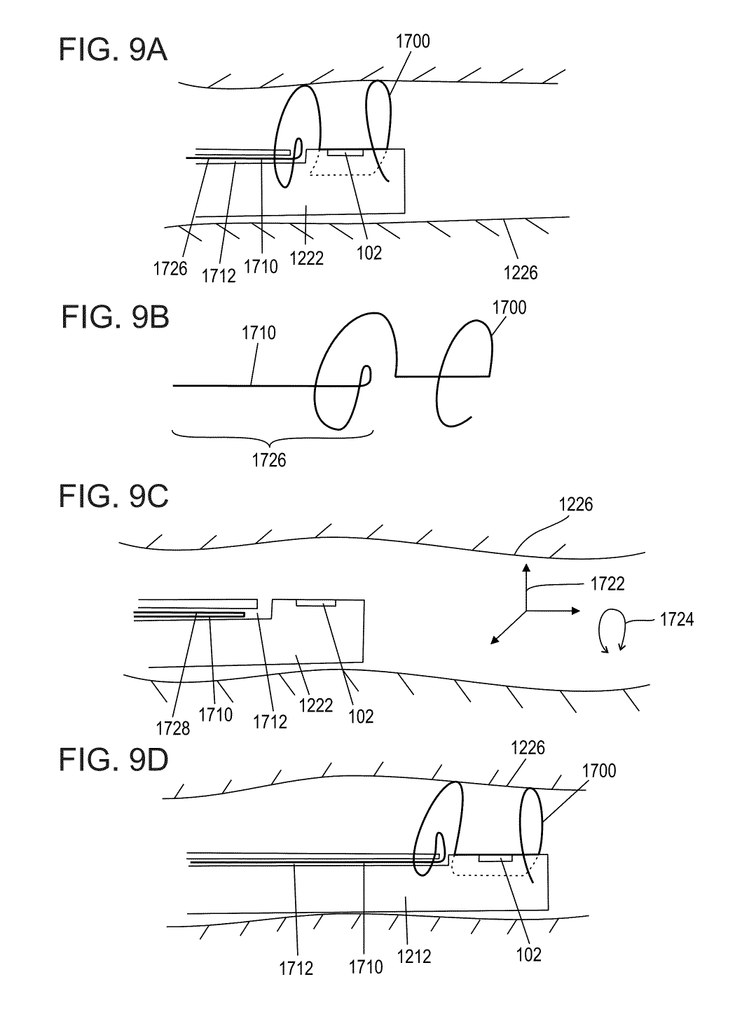

FIG. 8 is a method, illustrated by FIGS. 9A-D, of the use of a retractable separation element, for example wire 1710, to reposition and/or re-orient catheter 1222, such as during a medical procedure, in accordance with some embodiments of the invention.

FIG. 9A illustrates catheter 1222, anchored using the separation element (e.g., arc 1700) in the blood vessel, for example, as described with reference to FIGS. 5A-B, in accordance with some embodiments of the invention. In some embodiments, catheter 1222 is not anchored, for example, as described with reference to FIGS. 2A-B.

In some embodiments of the invention, arcs 1700 are shaped by wire 1710, for example, at a proximal end. Optionally, a straight portion 1726 of wire 1710 is substantially straight, for example, the portion other than arcs 1700.

In some embodiments of the invention, at least some of wire 1710 (e.g., straight portion) is located inside catheter 1222, for example, inside a wire lumen 1712. Optionally, at least some of wire 1710 is accessible, for example, straight portion 1726 is outside the body.

For clarity, FIG. 9B illustrates wire 1710 of FIG. 9A having been removed from catheter 1222. Optionally, wire 1710 is separable from and/or reinsertable into catheter 1222.

At 2312, in some embodiments of the invention, arcs 1700 are retracted, for example, by pulling the distal end of wire 1710. Optionally, arcs 1700 are retracted into catheter 1222, for example, into wire lumen 1712. Optionally, arcs 1700 deform into a substantially straight configuration, for example, due to the confines of wire lumen 1712.

FIG. 9C illustrates catheter 1222 with deformed arcs 1728 (e.g., in a relatively straight configuration), after retraction of arcs 1700 into wire lumen 1712, in accordance with some embodiments of the invention.

Optionally, at 2314, in accordance with some embodiments of the invention, catheter 1222 is repositioned and/or reoriented, for example, displaced in space 1722 (e.g., up/down, forward/backwards, sideways) and/or rotated 1724 (e.g., clockwise/counterclockwise).

Optionally, at 2316, in accordance with some embodiments of the invention, arcs 1700 are reformed, for example, by deformed arcs 1728 re-emerging from wire lumen 1712, such as by pushing the distal part of wire 1710. Optionally, catheter 1222 is re-anchored at a different location in the blood vessel by arcs 1700.

FIG. 9D illustrates catheter 1222 after reformation of arcs 1700 and/or repositioning of catheter 1222, for example, to treat another volume of tissue in the blood vessel wall 1226. In some embodiments of the invention, deformed arcs 1728 return to substantially the same configuration, for example arcs 1700.

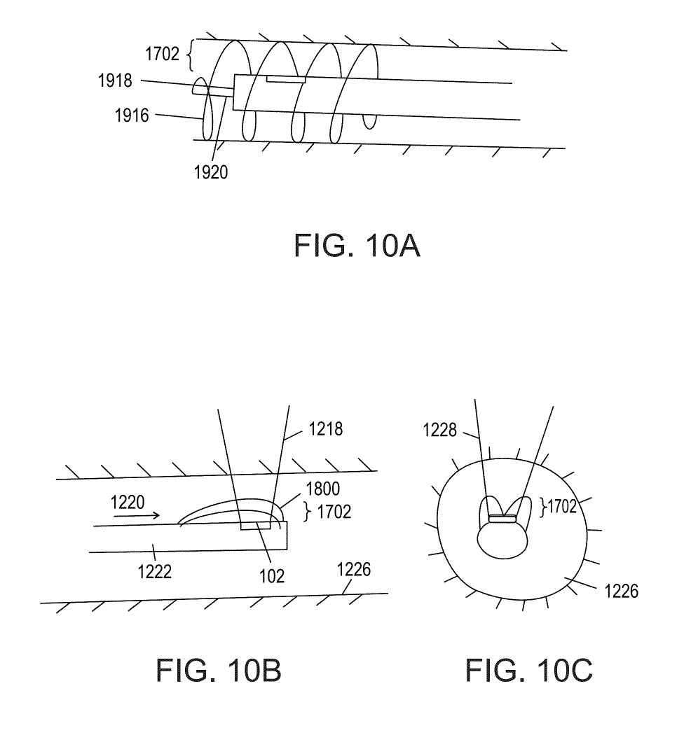

FIG. 10A illustrates some embodiments of the invention, wherein the retractable separation element is a helical shaped spring and/or coil 1916 coupled to guidewire 1920. Optionally, guidewire 1920 is threaded (e.g., pushed) through proximal end 1918 of catheter 1222, reforming coil 1916. Optionally, coil 1916 reforms in a distal direction, for example, over catheter 1222 and/or element 102. Alternatively, coil 1916 reforms in a proximal direction, for example, not surrounding a portion of catheter 1222.

A potential advantage of one or more of the above methods is preventing and/or reducing damage to wall 1226 (e.g., endothelium) during rotation and/or repositioning of catheter 1222, for example, by scraping the endothelium with arcs 1700.

### Exemplary Embodiment

### Longitudinal Arcs

FIGS. 10B (side) and/or 10C (cross sectional) illustrate the use of at least one to longitudinal arc 1800 (e.g., two arcs 1800) as the separation element, in accordance with some embodiments of the invention. Optionally, arc 1800 is arranged parallel to the long axis of element 102 and/or catheter 1222. Optionally, one end of arc 1800 is coupled proximally to element 102 and/or the other end is coupled distally to element 102.

### Exemplary Embodiment to Reduce Applied Force to Wall

FIG. 11A illustrates an embodiment of the separation element designed to reduce the force applied to wall 1226, in accordance with some embodiments of the invention. In the illustrated figure the separation element is not in contact with wall 1226.

In some embodiments of the invention, a free end 1804 of arc 1802 is not coupled to catheter 1222. Optionally, a second end of arc 1802 is coupled to catheter 1222, for example, proximal to element 102 (such as if arc 1802 is parallel to the long axis of catheter 1222 and/or element 102).

In some embodiments of the invention, free end 1804 does not contact catheter 1222, for example, a space 1816 of about 50 micrometers, about 100 micrometers, about 500 micrometers, or other smaller, intermediate or larger distances separates between end 1804 and catheter 1222. Alternatively, end 1804 contacts catheter 1222.

FIG. 11B illustrates arc 1802 of FIG. 11A contacting wall 1226, useful in practicing some embodiments of the invention.

Without being bound to theory, the applied force between wall 1226 and arc 1802 is transmitted into a deformation of arc 1802. A potential advantage is reducing the applied force to wall 1226, potentially reducing and/or preventing damage to wall 1226.

In some embodiments, space 1816 is reduced and/or eliminated (e.g., end 1804 contacts catheter 1222) by contact between arc 1802 and wall 1226. In some embodiments, end 1804 is displaced by contact, for example, sliding 1806 distally along catheter 1222.

### Exemplary Embodiment to Adjust Distance

FIGS. 12A-B illustrate some embodiments of a separation element for adjusting and/or setting the lower limit of the distance between element 102 and wall 1226 using an adjustable arc 1812. For example, a first distance 1808 is adjustable to a second distance 1810 (and/or vice versa).

In FIG. 12A adjustable arc 1812 (e.g., acts as the separation element) is made out of a wire 1814, in accordance with some embodiments of the invention. Optionally, one end of arc 1812 is coupled proximally relative to element 102. Optionally or additionally, a second end of arc 1812 and/or wire 1814 is controllable, such as by a physician from outside the body. Optionally, wire 1814 (e.g., portion that does not act as a separation element) is threaded through a lumen of catheter 1222.

In some embodiments, adjusting the distance occurs by pushing wire 1814 proximally, for example, from a free end of wire 1812.

FIG. 12B illustrates an embodiment of arc 1812 using wire 1814, having an end coupled distally relative to element 102. Optionally, the other end is threaded through a lumen in catheter 1222, having an opening proximal to element 102.

In some embodiments, increasing the distance occurs by pulling wire 1814 distally.

### Exemplary Embodiment

### In Situ Coupling to Catheter

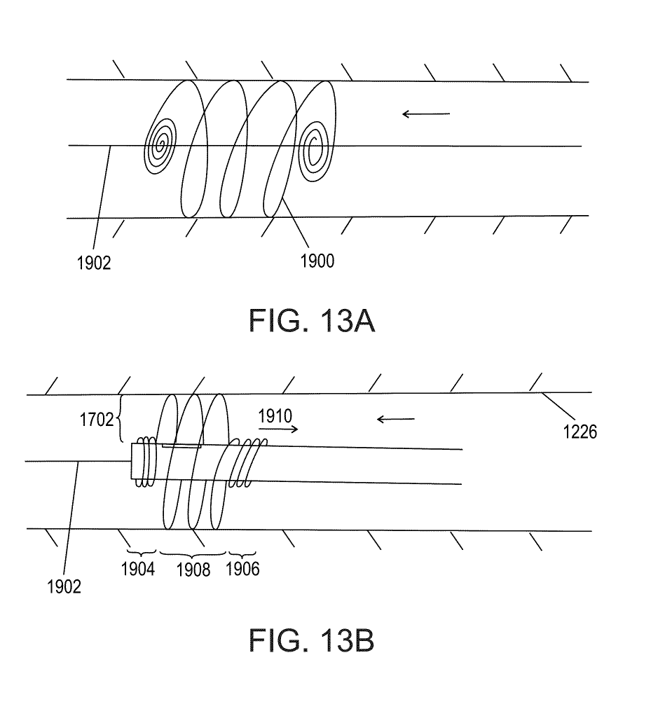

FIGS. 13A-B illustrate some embodiments wherein the separation device, such as a helical coil and/or spring 1900, is deployed in situ (e.g., inside the blood vessel). In some embodiments, catheter 1222 is coupled to spring 1900 while spring 1900 is in situ.

FIG. 13A illustrates spring 1900 that has been deployed in-situ, for example, through a guiding sheath. In some embodiments, a guidewire 1902 is threaded through the in-situ spring 1900.

As shown in FIG. 13B, catheter 1222 is coupled to spring 1900, for example, by being directed over guidewire 1902 and/or using tension. Optionally, coupling occurs at proximal region 1904 and/or distal region 1906. In some embodiments, catheter 1222 is anchored inside the blood vessel by spring 1900.

In some embodiments, the loops of spring 1900 at a first region 1904 and/or a second region 1906 are relatively closely spaced, for example, to have a relatively higher spring constant. In some embodiments, loops of spring 1900 at a third region 1908 are relatively widely spaced, for example, to have a relatively lower spring constant.

In some embodiments, upon contact of region 1908 with wall 1226 (e.g., applied to force), region 1908 expands, causing region 1906 and/or 1904 to slide 1910 longitudinally.

### EXEMPLARY EMBODIMENT FOR ROTATION OF THE ACOUSTIC ELEMENT

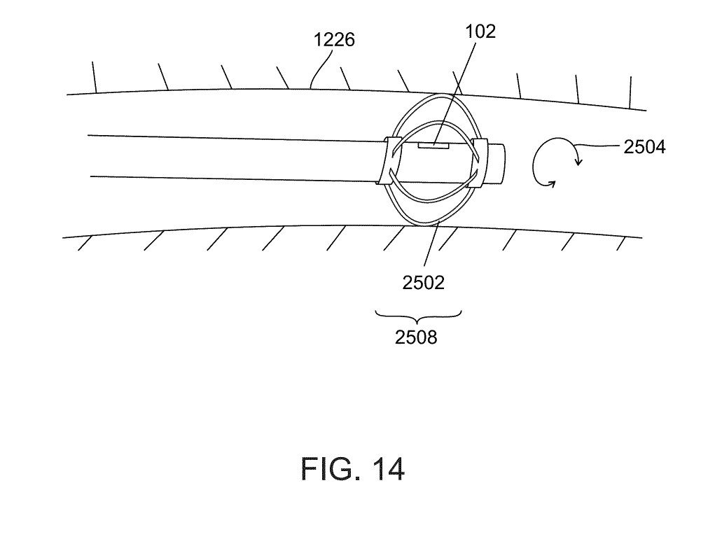

FIG. 14 illustrates some embodiments of the separation element, such as a plurality of arcs 2502, that provide for rotation 2504 and or movement of acoustic element 102 along the long axis of catheter 1222 (e.g., relative to wall 1226).

In some embodiments, arcs 2502 are coupled to an axle 2506 of catheter 1222, for example loosely coupled allowing catheter 1222 to rotate 2504 relative to axle 2506. Optionally, a distal end 2508 (e.g., comprising element 102) of catheter 1222 rotates relative to axle 2506. Alternatively or additionally, all of catheter 1222 rotates.

In some embodiments, arcs 2502 anchor end 2508 relative to wall 1226. A potential advantage is treating different volumes of tissue around the circumference of wall 1226 without having to reposition catheter 1222. Another potential advantage is reducing and/or preventing damage to wall 1226, by not rotating the separation element against wall 1226 and/or not having to reposition the separation element against wall 1226.

### Additional Non-Limiting Exemplary Embodiments

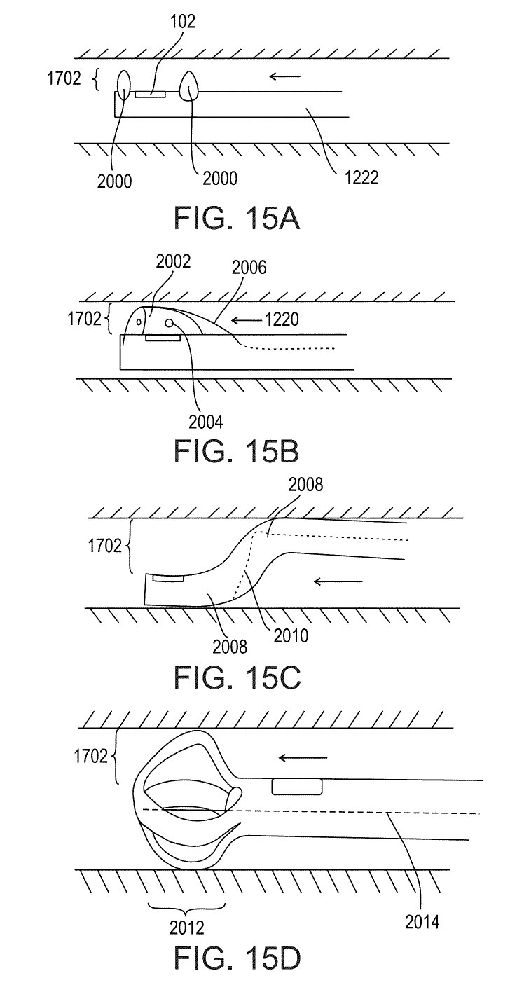

FIGS. 15A-D illustrate some additional non-limiting designs of the separation device, in accordance with some embodiments of the invention.

FIG. 15A illustrates the use of at least one balloon 2000, for example, inflated and/or deflated by saline. In some embodiments balloon 2000 is coupled to less than the circumference of catheter 1222, for example, balloon 2000 is coupled to catheter 1222 at an area proximal and/or distal to element 102. Alternatively, balloon 2000 is made out of a pliable material, for example, foam, as opposed to and/or in additional to being inflated and/or deflated.

FIG. 15B illustrates the use of a sheet 2002 to maintain distance 1702 by filling with flowing blood 1220, causing the distal end of catheter 1222 to ‘float’ away from wall 1266, for example, similar to a sail and/or parachute. One or more examples of materials for sheet 2002 include, nitinol, polymer such as polyurethane. Optionally, one or more holes 2004 in sheet 2002 maintain blood flow 1220 over element 102. Optionally, sheet 2002 is retractable, for example, by pulling a wire 2006.

FIG. 15C illustrates one or more bends 2008 and/or curvatures of catheter 1222 to maintain element 102 away from wall 1226. Optionally, bends 2008 are a permanent part of catheter 1222. Alternatively bends 2008 are controllable, for example, by one or more wires 2010. Optionally, the bending is controlled for several opening sizes. Optionally, the opening is monitored by using the radio-opaque markers on the bend zones, for example, by viewing the markers on fluoroscopic images.

FIG. 15D illustrates the use of an ellipsoid-like distal end 2012 of catheter 1222, formed for example, by making four cuts spaced about 90 degrees apart, and connecting the four free ends. Optionally distance 1702 is adjustable, for example, by adjusting the length of a perpendicular radius, such as by pulling a wire 2014.

### Exemplary Method of Calibration

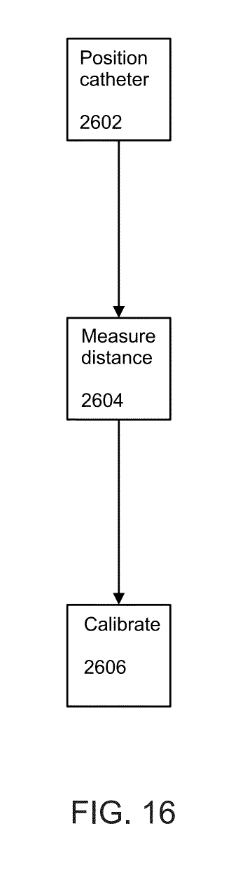

FIG. 16 is a flow chart of a method to use the separation device to configure and/or calibrate the ultrasound beam, such as for treatment of tissue in the vessel wall, in accordance with an exemplary embodiment of the invention.

Optionally, at 2602, the catheter (e.g., catheter 1222) is positioned inside the blood vessel using the separation element, in accordance with an exemplary embodiment of the invention. Optionally, the catheter (e.g., distal end) is anchored in the blood vessel using the separation element, in accordance with an exemplary embodiment of the invention. In some embodiments, the catheter is not anchored.

At 2604, the distance from the acoustic element to the vessel wall is estimated and/or measured. Optionally, the distance is measured by analyzing returning ultrasound echoes. Alternatively or additionally, the distance is estimated according to dimensions of the separation element, for example, if separation device is used to anchor the catheter, for example, if distance is adjustable, the adjustment can be estimated. Alternatively or additionally, the distance is estimated from x-ray images, such as fluoroscopic images taken during a medical procedure.

In some embodiments, the distance is estimated to be approximately zero (e.g., element touching vessel wall) versus greater than zero.

At 2606, the distance is used to calibrate the ultrasound control system, for example, by automatically calibrating a controller configured to control the ultrasound output (e.g., a look-up table and/or calculations). Alternatively, calibration is manual, for example, presented to a user to make a decision.

In some embodiments, calibration is binary, for example, ultrasound energy is generated if the distance is greater than zero and/or ultrasound energy is not generated if the distance is approximately zero.

In some embodiments, calibration is variable, for example, the intensity of the ultrasound energy produced (e.g., the maximum level) is based on the distance. A non-limiting example of a calibration table includes:

| | | | | | |
|---|---|---|---|---|---|
| Distance of acoustic element from wall | 0 | 0.5 | 1 | 1.5 | >=2 |
| [mm] | | | | | |
| Max acoustic intensity level [W/cm\hat{ }2] | 1 | 10 | 25 | 45 | 80 |

### Exemplary Element to Modify the Ultrasound Beam

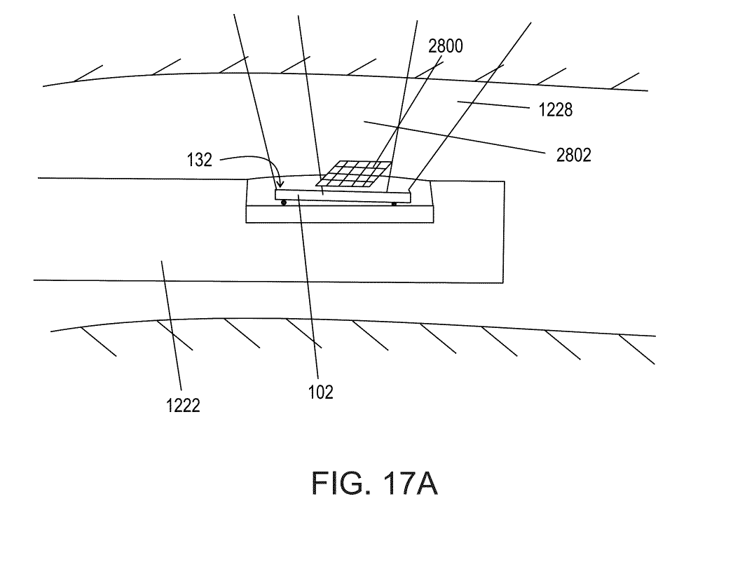

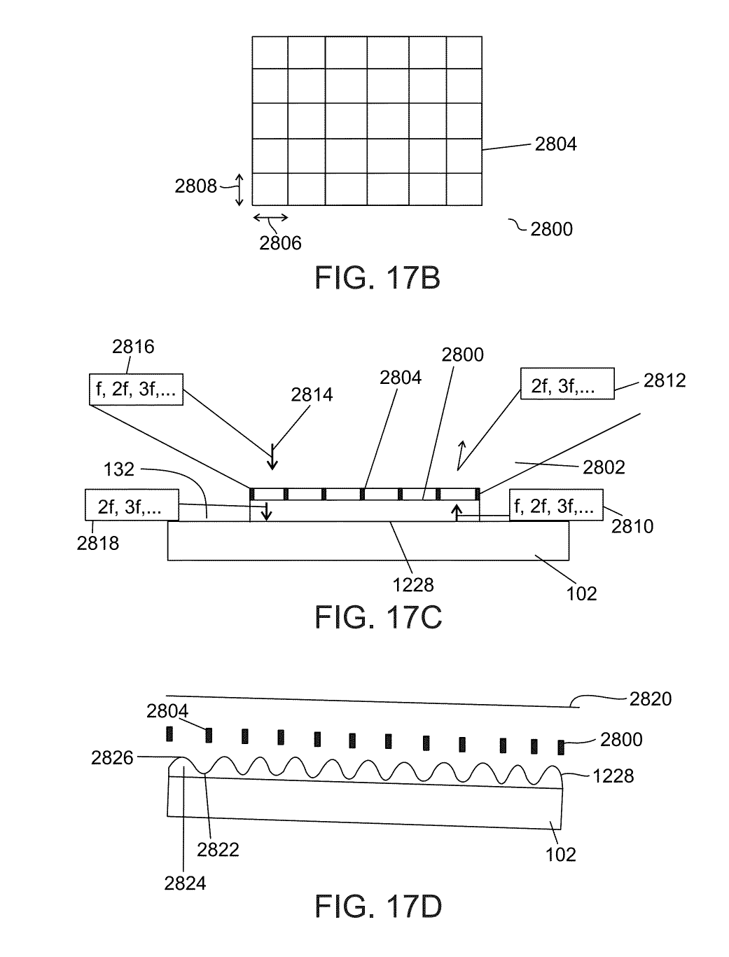

FIG. 17A illustrates an element designed to modify ultrasound beam 1228 produced by element 102, such as a lattice 2800, in accordance with an exemplary embodiment of the invention. In an exemplary embodiment of the invention, lattice 2800 modifies ultrasound beam 1228 to an affected US beam 2802.

In an exemplary embodiment of the invention, lattice 2800 modifies at least some of beam 1228, for example, by at least some surface 132 area of element 102. Alternatively, lattice 2800 overlays most and/or all surface 132 of element 102.

In an exemplary embodiment of the invention, lattice 2800 is suspended above surface 132 of element 102, for example, 100 micrometers above, 0.5 mm above, 1 mm above, or other smaller, intermediate or larger millimeters above. Alternatively or additionally lattice 2800 (e.g., or some part of lattice) contacts surface 132.

In an exemplary embodiment of the invention, lattice 2800 is made out of an ultrasonically reflective material, such as a metal, for example, stainless steel. A potential advantage is that lattice 2800 does not get hot, as the reflective material does not absorb the ultrasound energy.

FIG. 17B is a top view of a non-limiting example of lattice 2800, in accordance with an exemplary embodiment of the invention.

In an exemplary embodiment of the invention, lattice 2800 is made out of a grid of wires 2804.

In an exemplary embodiment of the invention, the diameter of wires 2804 is about 20 micrometers, about 60 micrometers, about 100 micrometers, or other smaller, intermediate or larger diameters are used.

In an exemplary embodiment of the invention, spacing 2806 between wires 2804 along the x-axis and/or spacing 2808 along the y-axis is designed to be approximately equal to the projected generated wavelength of ultrasound beam 1228 (e.g., according to the frequency of vibration of element 102).

FIG. 17C illustrates a side view of lattice 2800 modifying US beam 1228 to affected beam 2802, in accordance with an exemplary embodiment of the invention. For illustrative purposes, lattice 2800 is positioned approximately in the middle of surface 132, for example, over about 10% of surface 132, or over about 25%, over about 50%, over about 75%, over about 100%, over about 150%, over about 200%, or other smaller, intermediate or larger percentages relative to the area of surface 132. For clarity, only the US beam 1228 affected by lattice 2800 is illustrated.

In an exemplary embodiment of the invention, US beam 1228 is spread out by lattice 2800, for example. Optionally, beam 1228 is spread out to beam 2802, to have an effect similar to an ultrasound beam produced by the entire surface area 132, for example, similar to an ultrasound beam produced by element 102 without lattice 2800.

In an exemplary embodiment of the invention, lattice 2800 filters beam 1228. Optionally, the first harmonic of beam 1228 is filtered. For example, beam 1228 comprises harmonics 2810 such as f, 2f, 3f, etc. . . . where ‘f’ is the fundamental frequency of vibration of element 102. Lattice 2800 filters ‘f’ (e.g., by spacing 2806 and/or 2808 substantially equal to the wavelength of ‘f’), resulting in filtered harmonics 2812 such as 2f, 3f, etc. Alternatively or additionally, the first harmonic of a reflected beam 2814 (e.g., echo of ultrasound beam from tissues) is filtered, for example, from harmonics 2816 such as f, 2f, 3f, etc. . . . to harmonics 2818 such as 2f, 3f, etc.

A potential advantage of filtering the first harmonic is to use element 102 designed to produce a frequency ‘f’ for treatment and/or imaging at ‘2f’.

A potential advantage of lattice 2800 is to use the same element 102 for to treatment and/or imaging, for example, by positioning lattice 2800 approximately in the middle of element 102. Affected beam (e.g., beam 2802) is diverged to image a larger area and/or volume of tissue in the vessel wall. Beam 1228 produced by element 102 not overlapping with lattice 2800 can be used for treatment.

FIG. 17D illustrates a design of lattice 2800 to modify beam 1228 to have a substantially uniform US field 2820, in accordance with some embodiments of the invention.

In some embodiments, wires 2804 are positioned substantially at the local troughs 2822 of Hyugen wavelets 2824. Without being bound to theory, Inventors believe that wires 2804 remove local peaks 2826 of wavelets 2824, such as by spreading wavelets 2824.

It should be noted that the embodiment of lattice 2800 has been presented in a non-limiting manner, for example, more than one lattice 2800 can be used, for example, lattice 2800 can be non-uniform such as variable spacing 2806 and/or 2808.

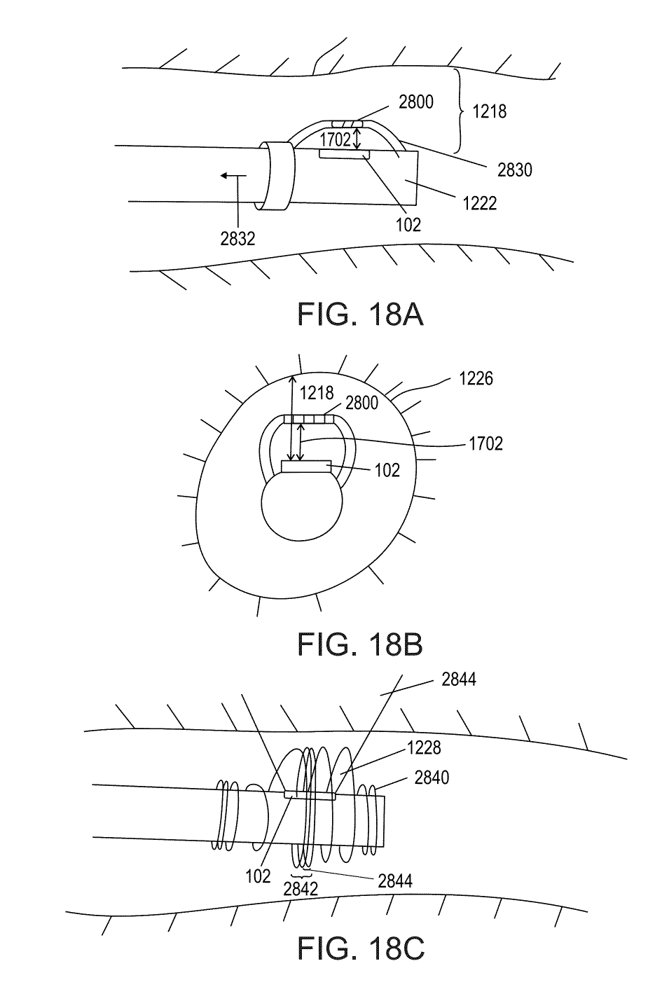

FIG. 18A is a side view, and FIG. 18B is a cross sectional view of the beam modification element (e.g., lattice 2800) used as a separation element, in accordance with an exemplary embodiment of the invention. Optionally, lattice 2800 maintains at least distance 1702 between element 102 and vessel wall 1226.

In some embodiments, lattice 2800 is positioned above element 102, for example, using two longitudinal wires 2830. Optionally, wires 2830 are coupled to catheter 1222 at one end, for example, proximally to element 102. Optionally or additionally, wires 2830 are coupled to catheter 1222 at a second end, for example, distally to element 102.

In some embodiments, wires 2830 are displaceable along the long axis of catheter 1222, for example, being able to slide distally 2832, such as to absorb and/or transfer a force during contact between lattice 2800 and wall 1226. Alternatively, sliding 2832 occurs to allow wires 2830 and/or lattice 2800 to fit inside a sheath, in accordance with an exemplary embodiment of the invention.

FIG. 18C illustrates a design of using a coil 2840 for the ultrasound beam to modification element, in accordance with some embodiments of the invention. Optionally, a beam modification portion 2842 of coil 2840 is used to modify the ultrasound beam. Optionally, coil 2840 acts as a separation device.

In some embodiments, a gap 2844 between loops of coil 2840 in beam modification portion 2842 is designed to filter and/or spread beam 1228, for example, gap 2844 is equal to about the wavelength produced by element 102 vibrating at the projected treatment frequency.

In some embodiments, coil 2840 is positioned over element 102 to result in a substantially uniformly affected US beam 2844, for example, positioned according to the generated Hyugen wavelets.

### Some Treatment Protocols According to Some Embodiments

In some embodiments of the invention, one or more actions are performed. Actions can be performed manually, such as by the user (e.g., cardiologist) and/or automatically such as by the control system. Optionally, actions are performed according to the measured and/or estimated distance between the ultrasonic emission element and the arterial wall.

In some embodiments of the invention, one or more protocols such as described in the table below are not affected by the vessel diameter, for example, if the vessel diameter is in the range of 3 and 8 mm for renal arteries, or even up to 50 mm for the aorta.

| | Distance | | | | | Action: |
|---|---|---|---|---|---|---|
| | (calculated/ | Value | | | Action: | System/ |
| | measured) | (mm) | Subject | Criterion | Cardiologist | algorithm |
| 1.1 | Distance | <1.4 | Catheter | >1 | Change catheter | System alert- |
| | measurement | | position | | position | short distance |
| | before | | before | | Change US | System disables |
| | treatment | | treatment | | transducer angle | excitation until |
| | | | | | Change US | distance is |
| | | | | | transducer position | changed |
| | | | | | along the artery | according to |
| | | | | | | criterion |
| 1.2 | | >1 | Catheter | >1 | Enable excitation | Enable |
| | | | position | | | excitation |
| | | | before | | | |
| | | | treatment | | | |
| 1.3 | | >5 | Catheter | 1 < x < 5 | Too large distance: | System alert- |
| | | | position | | Confirm possible | bifurcation, |
| | | | before | | bifurcation or | change US |
| | | | treatment | | dislocation with | transducer |
| | | | | | contrast injection | position |
| | | | | | and angiography | |
| | | | | | and move US | |
| | | | | | transducer | |
| 1.4 | | 1 > x > 1.3 | Catheter | >1 | Unreliable | |
| | | Angle | position | | distance-Confirm | |
| | | between | before | | US transducer | |
| | | US | treatment | | angle with contrast | |
| | | transducer | | | injection and | |
| | | and | | | angiography | |
| | | artery | | | Change catheter | |
| | | wall is | | | position | |
| | | larger | | | | |
| | | than 10° | | | | |
| 1.5 | | >1.3 | Catheter | >1 | Enable excitation | Enable |
| | | Angle | position | | | excitation |
| | | between | before | | | |
| | | US | treatment | | | |
| | | transducer | | | | |
| | | and | | | | |
| | | artery | | | | |
| | | wall is | | | | |
| | | larger | | | | |
| | | than 10° | | | | |
| | | (diagonal) | | | | |
| 2.1 | Distance | <1 | Catheter | >1 | Change catheter | System |
| | measurement | | position | | position and | alerts- |
| | during | | during | | complete treatment | short |
| | treatment | | treatment | | | distance |
| | | | | | | System |
| | | | | | | stops |
| | | | | | | excitation |
| | | | | | | until |
| | | | | | | distance is |
| | | | | | | changed |
| | | | | | | according |
| | | | | | | to |
| | | | | | | criterion |
| 2.2 | | 0.7 < x < 1 | Catheter | >1 | Consider to stop | System alert- |
| | | | position | | excitation and | shortening |
| | | | during | | improve position | distance |
| | | | treatment | | before completing | |
| | | | | | treatment | |
| 2.3 | | 1 < x < 5 | Catheter | >1 | Enable excitation to | Enable |
| | | | position | | end | excitation to |
| | | | during | | | end |
| | | | treatment | | | |
| 2.4 | | Decreasing | Catheter | >1 | Possible blood | System alert- |
| | | distance | position | | vessel constriction | decreased |
| | | | during | | due to treatment- | distance |
| | | | treatment | | consider stop and | |
| | | | | | nitroglycerin | |
| | | | | | infusion | |
| 2.5 | | >5 | Catheter | >1 | | System |
| | | Sudden | position | | | disabled |
| | | movement | during | | | |
| | | | treatment | | | |
| 3.1 | Vessel | Repetitive | Pulsation | | | Calculated blood |
| | blood | changes- | detection | | | pulsation |
| | pulsation | Maximal | before | | | If normal, |
| | analysis | position to | treatment | | | enable |
| | | minimal | | | | excitation |
| | | position | | | | |
| 3.2 | | No | Pulsation | | Possible no | System alert-no |
| | | differences | detection | | circulation: | pulsation, possible |
| | | between | before | | Validate flow by | constriction |
| | | Maximal | treatment | | contrast injection | |
| | | position to | | | and angiography | |
| | | minimal | | | If needed-infuse | |
| | | position | | | with nitroglycerin or | |
| | | | | | cold saline | |
| 3.3 | | distance>1 | Pulsation | >1 | Validate flow by | System alert-no |
| | | But no | detection | | contrast injection | pulsation, possible |
| | | pulsation | before | | and angiography | constriction |
| | | detection | treatment | | If needed-infuse | |
| | | | | | with nitroglycerin or | |
| | | | | | cold saline | |
| 4.1 | Distance | 1^{st}-<1 | Artery | | Possible | System alert- |
| | measurement, | 2^{nd}-<1 | diameter | | constriction: | possible |
| | rotate | | evaluation | | Confirm by contrast | constriction |
| | 180°, | | | | injection and | Disable excitation |
| | distance | | | | angiography | |
| | measurement | | | | If needed-inject | |
| | | | | | nitroglycerin and | |
| | | | | | treat | |
| 5.1 | Distance | <3 | Artery | | Check if US | System alert- |
| | measurement | | diameter | | transducer is located | possible |
| | in 4 | | evaluation | | in the correct artery- | constriction |
| | angles | | | | using contrast | Disable excitation |
| | (90°)- | | | | injection and | |
| | artery | | | | angiography | |
| | diameter | | | | Check for local | |
| | calculation | | | | constriction | |
| | | | | | Possible- | |
| | | | | | Nitroglycerin | |
| | | | | | injection | |
| 5.2 | | 3 < x < 8 | Artery | | Enable excitation | Enable excitation |
| | | | diameter | | | |
| | | | evaluation | | | |

Table of Some Possible Actions Based on the Distance from the Ultrasound Element to the Arterial Wall

### Potential Advantages of Some Embodiments

Further details of the system described herein can be found in the related applications. For example, “ULTRASOUND EMISSION ELEMENT” (attorney docket no. 52344) describes an ultrasound emission element. For example, “AN ULTRASOUND TRANSCEIVER AND USES THEREOF” (attorney docket no. 52345) describes a method for feedback and control. For example, “AN ULTRASOUND TRANSCEIVER AND COOLING THEREOF” (attorney docket no. 52346) describes cooling of the ultrasonic element. For example, “TISSUE TREATMENT” (attorney docket no. 52347) describes a method and an apparatus for tissue treatment. For example, “ULTRASOUND TRANSCEIVER AND USES IN DETECTION” (attorney docket no. 52342) describes ultrasonic imaging.

Some embodiments have one or more of the following exemplary advantages:
Relatively faster treatment, for example, a treatment duration of 5-30 seconds per treatment region, or other smaller, larger or intermediate ranges can be used.
Relatively small number of treatment regions per artery for renal denervation, for example, 4 treatment regions, or other smaller, intermediate or larger number of treatment regions is used.
Remote and/or localized effect, for example,
   Accurate control of the thermal effect and/or location, such as good control on the location and/or size of the artery tissue damage by therapeutic parameters.
   Ability to treat relatively large continuous areas in the arterial wall.
   A treatment option for short artery stumps and/or for short total treatment durations (e.g., 5-10 minutes vs 20 minutes for RF treatments).
   The thermal effect volume in the tissue is relatively far from the transducer face (e.g., media, adventitia, vasa-vasorum, peri-adventitia, adventitia nerves, peri-adventitia nerves, peri-adventitia capillaries). Targeting tissues in varying distances from the transducer face according to treatment parameters. For example, applying the thermal effect in tissues located about 5 mm or more from the lumen wall (e.g., intima layer). A relatively far effect is relevant for example, for achieving peripheral nerves blocks from inside the peripheral arteries.
   Non-targeted tissues on the beam path to the target tissue are not damaged and/or are selectively damaged (e.g. according to a margin of safety), for example, the endothelium, basal membrane and/or internal elastic lamina
   Possibility for varying levels of thermal modulation of the target tissue. For example, partial damage to nerves and/or other target tissues, in a controlled manner and different effect levels. Potentially, partial nerve injury can be controlled, that might lead to nerve recovery, either partially or entirely.
Tissue selectivity, for example, highly selective remote thermal effect in nerve bundles, such as nerves that are covered with thick fat tissue. For example as used in a Renal Denervation procedure in the Renal Artery ostium.
Treatment features suitable for Renal Denervation include:
   The ability to work very close to the renal artery ostium, for example, <10 [mm], or other smaller, intermediate or larger values.
   The ability to work in short arteries, for example, <20 [mm], or other smaller, intermediate or larger values
   The ability to work in small arteries, for example, 4-3 [mm], or other smaller, intermediate or larger values
Safety issues
   Relatively safer treatment.
   The temperature of the blood that flows over the ultrasonic transducer can be controlled to not exceed a temperature threshold of 50 degrees Celsius (or other smaller, intermediate or larger numbers) while working in the maximal allowed operation intensity level, for example, 50 [W/cm̂2], or other smaller, intermediate or larger intensity levels.
   The temperature of the blood that flows over the ultrasonic transducer can be controlled to not exceed a temperature threshold of over 43 degrees Celsius (or other smaller, intermediate or larger numbers), for example, while working in the therapeutic operation intensity level 30 [W/cm̂2], or other smaller, intermediate or larger intensity levels. In some embodiments, there is no need to add external cooling such as by saline injection.
   The therapeutic treatment on the blood vessel wall is done with no mechanical contact with the vessel wall, thereby reducing or eliminating the danger of damaging the vessel wall or disrupting any pathologies on the wall (e.g., atherosclerotic plaques). For example, reducing the risk of arterial perforation and/or mechanical damage that might cause a narrowing in the vessel, plaque tear and/or emboli.
   Localized and/or controlled effects specifically in the targeted treatment volume, preventing and/or reducing non-controlled energy effects in other tissues.
   Blocking of the blood flow during the treatment is optional, and in some embodiments, is not required.
   Treatment of a single artery location (e.g., longitudinally) in one or more circumferential directions, potentially, significantly reducing and/or preventing stenosis.
   Preventing and/or reducing damage to the artery due to repeating to treatment 2-3 times (or more) at the same region/direction, such as due to a mistake.
   Prevent and/or reduce interference with implanted electronic medical devices (e.g., pacemakers, defibrillators).
   Clinical implications, for example, relatively lower pain during treatment as a result of relatively faster blocking of nerves, with no electric excitation of the target nerve and/or no effect on other nerves. Potentially reducing sedation and/or anesthesia.
Relatively shallow learning curve, as leverages existing operator skill sets.
Many applications and/or ability to treat a wide range of clinical disorders.
Treatment option for a wide range of patients, such as high risk populations, for example as those suffering from vascular pathologies. Ability to treat in arteries with plaques and/or stents.
Ability to obtain a partial clinical effect (vs. complete effect). Potentially suitable for patients with milder disease, such as mild hypertension.
Feedback availability during treatment, such as information on the direction and location of the applied energy, catheter and the therapeutic catheter tip:
   Easy control capability and a clear direction and location of the ultrasonic ray and/or catheter location to carry out treatment, such as according to the ultrasonic echo reflection analysis.
   Ability to control the circumferential direction of the artery tissue damage.
   Continues information (e.g., ultrasonic measurement) on the position of the catheter tip, such as from the artery wall during treatment.
   Automatic detection of unwanted and/or risky movement of the catheter during treatment.

### General

It is expected that during the life of a patent maturing from this application many relevant ultrasound transducers will be developed and the scope of the term transducer is intended to include all such new technologies a priori.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for keeping an ultrasound emission element away from a wall of a blood vessel or cavity comprising:
inserting a catheter comprising the ultrasound emission element into a blood vessel or body cavity such that said ultrasound emission element is prevented from contacting a wall of said blood vessel or said cavity; and
maintaining a minimum distance between said ultrasound emission element and said wall to regulate flow of blood past said ultrasound emission element.

2. A method according to claim 1, wherein said preventing comprises preventing by a part of said catheter.

3. (canceled)

4. A method according to claim 1, wherein said minimum distance is at least one member of the group consisting of constant, adjustable and variable.

5.-6. (canceled)

7. A method according to claim 1, wherein said ultrasound emission element is a transducer, and/or is further adapted to receive ultrasound energy.

8. (canceled)

9. An apparatus for keeping an ultrasound emission element away from a wall of a blood vessel or cavity comprising:
an extension on a catheter comprising the ultrasound emission element, said extension sized and shaped to prevent contact between said ultrasound emission element and a wall of a blood vessel or cavity and to maintain a minimum distance between said ultrasound emission element and said wall.

10. An apparatus according to claim 9, wherein said extension is transparent to ultrasound energy emitted by said ultrasound emission element.

11. An apparatus for keeping an ultrasound emission element away from a wall of a blood vessel or cavity comprising:
a catheter comprising the ultrasound emission element; and
a sheath, said catheter guided inside said sheath into a blood vessel or a lumen, and wherein said sheath is configured to prevent contact between said ultrasound emission element and a wall of a blood vessel or cavity and to maintain a minimum distance between said ultrasound emission element and said wall.

12. An apparatus for keeping an ultrasound emission element away from a wall of a blood vessel or cavity comprising:
a deformable catheter comprising the ultrasound emission element, said catheter configured to deform with a predetermined deformation, said deformation configured to prevent contact between said ultrasound emission element and a wall of a blood vessel or cavity and to maintain a minimum distance between said ultrasound emission element and said wall, thereby to regulate blood flow along said wall and provide cooling.

13. An apparatus according to claim 12, wherein said deformation is configured to change between a first state, said first state comprises preventing contact between said ultrasound emission element and said wall, and a second state, said second state allowing contact between said ultrasound emission element and said wall.

14. A catheter for medical therapy comprising:
a distal end; and
a proximal end comprising:
an ultrasound emission element; and one member of the group consisting of:
one or more wires, at least one end of said one or more wires coupled at least proximal or distal to said ultrasound emission element, said one or more wires forming a substantially looping shape, and a dimension of said looping shape sufficient to prevent contact between said ultrasound emission element and a vessel wall; and
one or more positioning elements, at least one end of said one or more positioning elements coupled at least proximal or distal to said emission element, and a dimension of said one or more positioning elements sufficient to prevent contact between said emission element and a vessel wall, and to control the flow of blood between said ultrasound emission element and said wall; and
one or more sheets, at least one edge of said one or more sheets coupled at least proximal or distal to said ultrasound emission element, said sheets expand sufficiently to prevent contact between said ultrasound emission element and a vessel wall;
said member further configured to regulate the flow of blood between said ultrasound emission element and said wall by setting a minimum distance between said ultrasound emission element and said wall, thereby to regulate cooling of said wall.

15. A catheter according to claim 14, wherein said one or more wires are arranged along the long axis of said catheter, or wherein said one or more wires are arranged perpendicular to the long axis of said catheter, or wherein said one or more wires are arranged along the circumference of said catheter, or wherein a first end of said one or more wires is coupled to said catheter and a second end of said one or more wires is not coupled to said catheter.

16.-18. (canceled)

19. A catheter according to claim 14, wherein said dimension of said looping shape is adjustable, or wherein said dimension of said one or more wires is sufficiently large to anchor said catheter inside a blood vessel, or wherein said one or more wires are not substantially within an ultrasound beam produced by said ultrasound emission element, or wherein said one or more wires do not substantially block a flow of blood over said ultrasound emission element, or wherein a diameter of said one or more wires is between 30 micrometers and 200 micrometers.

20.-24. (canceled)

25. A catheter according to claim 14, wherein said proximal end of said catheter is positioned along a long axis of said one or more positioning elements, or wherein said one or more positioning elements are a spiral, or wherein said one or more positioning elements are positioned around an outer circumference of said proximal end of said catheter, or wherein said one or more positioning elements comprises one or more springs, or wherein said one or more spring comprises at least two regions having at least two spring constants, or wherein said positioning elements are made of nitinol, or wherein said positioning elements have a shape memory in a way that is bent when in a guiding catheter but reshaped to an original shape in said vessel.

26.-31. (canceled)

32. A catheter as in claim 14, further comprising an open or close mechanism, or further comprising an open or close mechanism wherein said mechanism comprising a wire that mechanically opens or closes said wire by pulling or pushing said wire, or further comprising a mechanism comprising a sheath that mechanically opens or closes said positioning elements by covering or uncovering said memory shaped positioning elements.

33.-36. (canceled)

37. A catheter as in claim 14, wherein said member is said one or more sheets, the catheter further comprising an open or close mechanism, wherein said mechanism comprising a wire that mechanically opens or closes said sheet, or wherein said mechanism comprises one or more opening stages, said stage adapted to a diameter of said vessel.

38. (canceled)

39. A catheter for medical therapy comprising:
a distal end; and
a proximal end comprising:
an ultrasound emission element; and
one member of the group consisting of:
said proximal end of said catheter adapted to a substantially ellipsoid shape, and a dimension of ellipsoid sufficient to prevent contact between said ultrasound emission element and a vessel wall;
a device to anchor said proximal end inside a blood vessel, said device comprising an axle, said axle adapted to provide rotation of said catheter without rotation of said device and said device configured to prevent contact between said emission element and a wall of said vessel; and
one or more wires, at least one end of said one or more wires coupled at least proximal or distal to said ultrasound emission element, said one or more wires forming a substantially looping shape, and a dimension of said looping shape sufficient to prevent contact between said ultrasound emission element and a vessel wall;
said member configured to regulate the flow of blood between said ultrasound emission element and said wall to provide cooling to said wall, said regulating comprising setting a minimum distance between said ultrasound transmission element and said wall.

40. (canceled)

41. The catheter of claim 39, wherein said member is said one or more wires, and further comprising:
a modification element coupled to said one or more wires, said modification element having a spacing of about half a wavelength of a frequency of an ultrasound beam, and said modification element configured to at least one of spread and filter said ultrasound beam.

42. A catheter as in claim 41, wherein said modification element is one member of the group consisting of:
being positioned over at least some surface of said ultrasound emission element;
a lattice; and
a coil.

43.-44. (canceled)

45. A method of using a guiding sheath to repositioning an ultrasound catheter coupled to a separation element comprising:
deforming a separation element coupled to a catheter to a space between a guiding sheath and said catheter;
repositioning said catheter; and
reforming said separation element.

46. A method of using a retractable separation element to repositioning an ultrasound catheter comprising:
retracting a separation element;
repositioning said catheter; and
reforming said separation element.

47. A catheter for medical therapy comprising:
a distal end; and
a proximal end comprising:
an ultrasound emission element; and
a braiding cylinder from said proximal end, said cylinder sufficient to prevent contact between said ultrasound emission element and a vessel wall.

48. A catheter for medical therapy comprising:
a distal end; and
a proximal end comprising:
an ultrasound emission element; and
a balloon, said balloon having a volume sufficient to prevent contact between said ultrasound emission element and a vessel wall without blocking blood flow between said ultrasound emission element and said vessel wall.
